(19) United States
(12) Patent Application Publication
(10) Pub. No.: US 2003/0235563 A1
(43) Pub. Date: Dec. 25, 2003

(54) Placental derived stem cells and uses thereof

(76) Inventors: Stephen C. Strom, Allison Park, PA (US); Toshio Miki, Pittsburgh, PA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/420,656

(22) Filed: Apr. 21, 2003

Related U.S. Application Data
(60) Provisional application No. 60/374,172, filed on Apr. 19, 2002.

(51) Int. Cl. A61K 48/00; C12N 5/08

(52) U.S. Cl. 424/93.21; 435/370

(57) ABSTRACT

The present invention features novel placental derived stem cells and provides methods and compositions for the therapeutic uses of placental derived stem cells or placental derived stem cells that have been induced to differentiate into a desired tissue type into a recipient host in amounts sufficient to result in production of the desired cell type, i.e., hepatic, pancreatic, neuronal, or nervous tissue.

### 1. INTRODUCTION

The present invention provides novel placental derived stem cells capable of differentiating into a variety of different cell types. The invention also provides methods for prolonged culturing of placental derived stem cells with the capacity for differentiation into a variety of different cell type. The methods and compositions of the invention provide stem cells, or stem cells that have been induced to differentiate, that may be used in transplantation, development of bioartificial organs or drug screening assays designed to test the effectiveness and safety of drugs.

### 2. BACKGROUND OF INVENTION

Embryonic stem cells have long been recognized as a source of totipotent stem cells, able to give rise to different cell types. These cells are derived from the inner cell mass of fertilized and developing embryos. The use of such cells has been controversial on both ethical and religious grounds. Furthermore, federal regulation currently limits the use of embryonic stem cells to a few established cell lines which are difficult to obtain. Recent studies have focused on alternative sources of stem cells. These include hematopoietic stem cells obtained from bone marrow or peripheral blood. However the isolation of such stem cells from individuals can be invasive and painful.

The developing embryo requires the interaction between mother and embryo mediated by the placenta and extraembryonic membranes for survival. The placenta and chorion is derived from the trophoblast, which begins to differentiate from the inner cell mass as early as day 8 following fertilization while the amniotic cavity originates in the ectoderm of the inner cell mass and consists of a single layer of extraembryonic mesoderm.

In recent years, the placenta, the amnion and cord blood have been studied as an alternative sources of stem cells. The amnion is a structure comprised of a single layer of epithelial cells which completely surrounds the fetus as it develops in the uterus. Amniotic epithelial (AE) cells have unique features which make them potentially useful for cell transplantation. One such feature is the failure of AE cells to express MHC surface antigens on their cell surface (Akle et al., I: Immunogenicity of Human Amniotic Epithelial Cells after Transplantation into Volunteers. The Lancet 1003-1005, 1981; Sakuragawa et al., Immunostaining of Human Amniotic Epithelial Cells: Possible Use As A Transgene Carrier In Gene Therapy For Inborn Errors of Metabolism. Cell Transplantation 4:343-346, 1995; Tohyama et al., Characterization of Human Amniotic Epithelial Cells Transformed with Origin-Defective SV40 T-Antigen Gene. Tohoku J. Med. 182:75-82., 1997).

The absence of MHC expression on the surface of AE cells indicated that such cells might be useful in transplantation therapy. For example, Akle et al. reported that grafts of amniotic tissue were tolerated by 7 volunteers for times up to 54 days without evidence of rejection (Akle et al., I: Immunogenicity of Human Amniotic Epithelial Cells after Transplantation into Volunteers. The Lancet 1003-1005, 1981). In addition, several clinical trials of amniotic tissue transplantation have been carried out in patients with inborn errors in metabolism. Although a definitive clinical benefit following transplantation was not observed, a graft-versus-host reaction was not described in any patients with the possible exception of a single patient which was transplanted for Neimann-Pick disease type B (Scaggiante et al., Successful therapy of Nieman-Pick disease by implantation of human amniotic membrane. Transplantation 44: 59-61, 1987).

The most significant problem preventing the use of placental-derived amniotic derived cells in transplantation is the difficulty associated with their long-term propagation in culture, as well as reliable and reproducible mechanisms to induce differentiation of placental-derived cells into hepatocytes or other desired cell types. The successful recovery of placental-derived cells is often variable and dependent on the starting material.

Terada et al., reported that supplementation of a basal media which contained 10% FBS with either hepatocyte growth factor (HGF, 50 ng/ml) or epidermal growth factor (EGF, 50 ng/ml) increased the number of cells which could be obtained from an initial culture 2 to 7 fold. However, after 11 days of culture, no proliferative response was seen even in the presence of HGF or EGF and FBS (Terada et al., Inducing Proliferation of Human Amniotic Epithelial (HAE) Cells for Cell Therapy. Cell Transplantation 9:701-704, 2000).

In an attempt to prolong the replicative phase, aminiotic epithelial cells have been immortalized with SV40 Large T antigen. Although the cell line grew well, it had only limited experimental value because the cells were tumorigenic upon transplantation (Tohyama et al., Characterization of Human Amniotic Epithelial Cells Transformed with Origin-Defective SV40 T-Antigen Gene. Tohoku J. Med. 182:75-82., 1997).

Hu et al. has shown the isolation, culturing and cryopreservation of amniotic epithelial cells by the removal of the amnion from the placenta, and the mechanical or enzymatic removal of the amniotic epithelial cells (WO00/73421). They were able to freeze and thaw the cells, but did not demonstrate prolonged culturing of the cells or expression of any embryonic, pluripotent stem or differentiated cell markers.

Other investigators have described the isolation of fetal mesenchymal cells and potentially fetal mesenchymal stem cells from both the human placenta and amniotic fluid for use in fetal tissue engineering (Kaviani et al., The Amniotic Fluid as a Source of Cells for Fetal Tissue Engineering. J. Pediatr Surg. 36:1662-1665, 2001, Kaviani et al., The Placenta as a Cell Source in Fetal Tissue Engineering. J. Pediatr Surg. 37:995-999, 2002).

Using immunohistochemical techniques, Sakuragawa et al. demonstrated the expression of markers for both neuronal and glial cells on AE cells (Sakuragawa et al., Expression of Markers for Both Neuronal and Glial Cells in Human Amniotic Epithelial Cells. Neuroscience Lett. 209:9-12, 1996). Such neuronal markers include neurofilament protein (NF), microtubule associated protein 2 (MAP2), MAP2 kinase, glial fibrilliary acidic protein (GFAP) and cyclic nucleotide phosphodiesterase. Later studies also indicated that the AE cells express choline acetyltransferase mRNA and synthesize and release acetylcholine (Sakuragawa et al. Evidence for active acetylcholine metabolism in human amniotic epithelial cell: applicable to intracerebral allografting for neurological disease. Neurosci Lett. 232:53-56, 1997). Evidence for active acetylcholine metabolism in human amniotic epithelial cell: applicable to intracerebral allografting for neurological diseases like dementia. (Neurosci. Lett. 232:53-56,1997; EP815867) and catecholamines (Elwan and Sakuragawa, Evidence for synthesis and release of catacholamines by human amniotic epithelial cells. Neuroreport 8: 3435-3438, 1997). Evidence of AE liver cell specific protein expression has also been reported. For example, cultured AE cells were shown to be immunoreactive with antibodies to human albumin (Alb) and alpha-fetoprotein (AFP) in vitro and also following transplantation into mouse liver (Sakuragawa et al., Human Amniotic Epithelial Cells are Promising Transgene Carriers for Allogeneic Cell Transplantation Into Liver. J Hum Genet 45:171-176, 2000).

Additionally, Kobayashi et al., isolated amniotic epithelial cells and mesenchymal cells from human amniotic membranes that were predominantly cytokeratin-positive cells. These cells were characterized for their ability to inhibit neovascularization and were thought to contain potent inhibitors of neovascularization. The use of these cells in the treatment of corneal diseases with neovascularization was proposed in Kobayashi et al., (Suppression of corneal neovascularization by culture supernatant of human amniotic cells. Cornea 21:62-67, 2002).

Sackier et al, isolated amniotic epithelial cells to be applied to clinical procedures that include the treatment of diseased or damaged tissues, e.g. in joints denuded of cartilage and vascular grafts (U.S. Pat. No. 5,612,028, EP333328).

Some groups have also focused on the collection of other stem cell-like cells through exsanguination and subsequent perfusion of the placental tissue (US2002/0123141, US2003/0032179). These cells are isolated mainly from cord blood. They express hematopoietic stem cell markers and are believed to be pluripotent (US2002/0123141, US2003/0032179). However, reports have shown that these cells fuse with differentiated cells from tissues, rather than exhibiting pluripotentcy and the capability of differentiation into the tissue type (Wang et al. “Cell fusion is the principal source of bone-marrow-derived hepatocytes,” Nature, Mar. 30, 2003, electronic publication; Vassilopoulos et al., “Transplanted bone marrow regenerates liver by cell fusion,” Nature, Mar. 30, 2003, electronic publication.)

Accordingly, there is a need for an alternative, non-controversial source of stem cells that can differentiate into various tissues, including liver, pancreas, endothelial and nervous tissue and thereby provide useful therapeutics.

### 3. SUMMARY OF THE INVENTION

The present invention features novel placental derived stem cells (e.g. stem cells derived from the amnion, chorion or decidua of a placenta). Preferred cells are obtained from a human placenta. Other preferred placental derived stem cells express a biomarker selected from the group consisting of: c-kit, Thy-1, OCT-4, SOX2, hTERT, SSEA1, SSEA3, SSEA4, TRA1-60 and TRA1-81. In addition the cells are normally negative for expression of CD34. Particularly preferred cells are those deposited with American Type Culture Collection on ______ and assigned ATCC accession number ______. Other preferred placental derived stem cells have been genetically engineered to express an effective amount of a therapeutic protein.

The present invention further provides methods for deriving enriched populations of placental derived stem cells utilizing antibodies that recognize cell surface expressed stem cell markers. Such methods include the use of fluorescence activated cell sorting (FACS) to detect placental stem cells expressing specific cell surface markers.

The invention further relates to the in vitro attachment of placental-derived cells to a matrix prior to transplantation for the purpose of increasing the viability and growth of the transplanted cells. In addition, the matrix may be composed of additional materials including other types of cells or biologically active molecules.

In another aspect, the invention provides methods for culturing the placental derived stem cells to propagate the cells and for differentiating. In one embodiment, the cells are cultured under appropriate conditions and for a sufficient period of time to differentiate into hepatocytes. Hepatocyte like cells so derived have been found to express at least one marker selected from the group consisting of: albumin, CYP3A4, A1AT, HNF1, HNF4 and C/EBP-alpha. Particularly preferred hepatic-like cells are those deposited with American Type Culture Collection on ______ and assigned ATCC accession number ______. An effective amount of hepatocytes so derived may be administered to a subject to treat a liver disease.

In another embodiment of the invention, the hepatocytes may be used to form bioartificial livers for use by subjects having liver disease. Additionally, the stem cells may be used to form humanized animal livers that may be used as a bioartificial liver. The use of such bio-artificial livers involves the perfusion of the subject's blood or plasma through the bio-artificial liver. In the blood perfusion protocol, the subject's blood or plasma is withdrawn and is contacted with the hepatocyte cell cultures. During such passage, molecules dissolved in the patient's blood or plasma, such as bilirubin, are taken up and metabolized by the hepatocyte cultures. In addition, the cultured hepatocytes will provide factors normally supplied by liver tissue.

Alternatively, the hepatocyte-like cells may be useful in drug toxicity assays.

In another aspect, the invention provides for methods for culturing the placental derived stem cells under appropriate conditions and for a sufficient period of time to induce vascular endothelial cell differentiation. An effective amount of the vascular endothelial like cells so derived from placental stem cells in an effective amount, may be administered to a subject to treat a vascular disease.

In another aspect, the invention provides methods for culturing placental derived stem cells under appropriate conditions and for a sufficient period of time to induce pancreatic cell differentiation. Particularly preferred pancreatic-like cells are those deposited with American Type Culture Collection on ______ and assigned ATCC accession number ______ An effective amount of the pancreatic cells may then be administered to a subject to treat a pancreatic disease.

In a further aspect, the invention provides for methods for culturing the placental derived stem cells for a sufficient period of time to induce differentiation into cells of nervous tissue. An effective amount of the neuronal cells may be administered to a subject to treat a disease or disorder of the nervous system.

Placental derived stem cells provide a noncontroversial source of stem cells that can be differentiated into various tissues, including liver, pancreas, endothelial and nervous tissue. Other features and advantages of the invention will be apparent from the following Detailed Description and claims.

### 4. BRIEF DESCRIPTION OF THE DRAWINGS

FIG. 1 shows the source of various cell types isolated from a placenta.

FIG. 2 shows light micrographs of a cross section through a human placenta with the amnion, chorion and decidual layers are labeled. The insert shows a higher magnification of the amniotic membrane and its supportive stromal layer of mesenchymal cells.

FIG. 3 shows RT-PCR analysis of adherent and nonadherent cells derived from a placenta expressing stem cell marker, Oct-4, and a neuronal stem cell marker, SOX-2.

FIG. 4 shows FACS analysis of cultured placental-derived cells expressing embryonic antigens, SSEA-3 and SSEA-4.

FIG. 5 shows light micrographs of placental-derived cells isolated from the same placenta using isolation method and culture medium as described in Sakuragawa (FIG. 5a) or the isolation method and culture medium of the present invention (FIG. 5b).

FIG. 6 is a bar graph showing the relative differences in RNA expression of various liver-specific markers in placental-derived cells isolated using the method and culture medium as described in Sakuragawa or the isolation method and culture medium of the present invention.

FIG. 7 shows immunohistochemical staining of placental-derived cells with antibodies against AE1/AE3, CK19, CK18, c-kit, Thy-1, A1AT, AFP in human placental tissue and cultured cells.

FIG. 8 shows placental-derived cell expression of alkaline phosphatase (a, b) and human serum albumin (c-f) in human placental tissue and cultured cells.

FIG. 9 shows expression of albumin mRNA (a), albumin protein (b), and alpha 1 anti-trypsin protein (c) in placental-derived cells.

FIG. 10 shows immunohistochemical staining of placental-derived cells with antibodies against human HNF-4 in human hepatocytes (a) and placental-derived cells (b).

FIG. 11 is bar graph showing the relative differences in RNA expression of human albumin in cultured placental-derived cells cultured in various culture medium.

FIG. 12 is a bar graph showing the relative differences in RNA expression of CYP3A4 in cultured placental-derived cells cultured in various culture medium.

FIG. 13 is a bar graph showing the relative differences in RNA expression of A1AT in cultured placental-derived cells cultured in various culture medium.

FIG. 14 is a bar graph showing the relative differences in RNA expression of C/EBP alpha in cultured placental-derived cells cultured in various culture medium.

FIG. 15a is a bar graph showing that cultured placental-derived cells exhibit CPY1A1/CPY1A2 activity upon beta-napthoflavone induction. FIG. 15b shows an high pressure liquid chromatographic (HPLC) separation of testosterone metabolites generated in placental-derived hepatocytes.

FIG. 16a is a fluorescent micrograph of transplanted fluorescent-GFP-expressing placental-derived cells incorporated into a mouse liver. FIG. 16b is a 400× micrograph of the cells in FIG. 16a.

FIG. 17 is a micrograph of a mouse liver section showing transplanted placental-derived cells incorporated into an immunodeficient mouse liver expressing human alpha-1-antitrypsin.

FIG. 18 is a micrograph of a mouse liver section showing transplanted placental-derived cells incorporated into an immunodeficient mouse liver expressing human albumin.

FIG. 19 shows fluorescent micrographs showing cultured placental-derived cells expressing neuronal cell markers, GFAP, beta-tubulin III, and CNP.

FIG. 20 shows light and electron micrographs of cultured placental-derived cells cultured on matrigel that demonstrate characteristics of vascular endothelial cells.

FIG. 21 shows the results of RT-PCR analysis of cultured placental-derived cells expressing pancreatic islet cell markers, Pax6, insulin, Pdx1, Nkx-2,2 and glucagon.

### 5. DETAILED DESCRIPTION OF THE INVENTION

The present invention features novel placental derived stem cells that can be obtained from the amnion, chorion or decidual layers of the placenta. Exemplary cells were deposited with American Type Culture Collection, 10801 University Blvd. Manassas, Va. 20110-2209 ______, 2003 and have been assigned ATCC accession number ______. The placental derived stem cells of the invention express markers normally associated with embryonic stem cells including but not limited to c-kit, Thy-1, OCT-4, SOX2, hTERT, SSEA1, SSEA3, SSEA4, TRA1-60 and TRA1-81.

In addition, these placental derived stem cells have been found to be capable of differentiating into a variety of tissue types including but not limited to hematopoetic, liver, pancreatic, nervous and endothelial tissues. Such cells are particularly useful to restore function in diseased tissues via transplantation therapy or tissue engineering, and to study metabolism and toxicity of compounds in drug discovery efforts.

In an exemplary embodiment of the invention, placental derived stem cells are administered to a subject in need of new tissue or metabolic repair. Placental derived stem cells may be transplanted directly into the recipient where the cells will proliferate and differentiate to form new tissue thereby providing the physiological processes normally provided by that tissue. Alternatively, placental derived stem cells may be transplanted as a differentiated cell population.

The placental derived stem cells of the invention may also be used to humanize animal organs. Example 12 demonstrates transplantation of the stem cells of the invention into mouse liver and data showing the differentiation of the cells into human hepatocytes within the mouse liver. By this mechanism, human placental derived stem cells may be transplanted into an animal organ such as liver, pancreas or brain. The animal organ may or may not be depleted of its native cells prior to the transplant. The stem cells could be used to regenerate and repopulate the animal organ to reconstitute the animal organ with human functions. “Humanized” organs of such animals as mouse, rat, monkey, pig or dog could be useful for organ transplants into people with specific diseases. Humanized animal models may also be used for diagnostic or research purposes relating but not limited to, drug metabolism, toxicology studies and for the production study replication and therapy of viral or bacterial organisms. Mice transplanted with human hepatocytes forming chimeric human livers are already being used for the study of hepatitis viruses, which only grow in human hepatocytes (Dandri M et al. Repopulation of mouse liver with human hepatocytes and in vivo infection with hepatitis B virus. Hepatol. 33:981-988, 2001, and Mercer D F et al. Hepatitis C virus replication in mice with chimeric human livers. Nature Med. 7:927-933, 2001).

The placental derived stem cells or cells differentiated therefrom can be injected or implanted into target sites in the subjects, preferably via a delivery device, such as a tube, e.g., catheter, for injecting cells and fluids into the body of a recipient subject. In a preferred embodiment, the tubes additionally have a needle, e.g., a syringe, through which the cells of the invention can be introduced into the subject at a desired location. The progenitor cells of the invention can be inserted into such a delivery device, e.g., a syringe, in different forms. For example, the cells can be suspended in a solution or embedded in a support matrix when contained in such a delivery device. As used herein, the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating progenitor cells as described herein in a pharmaceutically acceptable carrier or diluent and, as required, other ingredients enumerated above, followed by filter sterilization.

In addition, placental derived stem cells may be attached in vitro to a natural or synthetic matrix that provides support for the cells prior to transplantation. The matrix will have all the features commonly associated with being biocompatible, in that it is in a form that does not produce an adverse, or allergic reaction when administered to the recipient host. Growth factors capable of stimulating the growth and regeneration of, for example, liver, pancreatic or neurological tissue may also be incorporated into matrices. Such matrices may be formed from both natural or synthetic materials and may be designed to allow for sustained release of growth factors over prolonged periods of time. Thus, appropriate matrices will both provide growth factors and also act as an in situ scaffolding in which the placental derived stem cells differentiate and proliferate to the new tissue of interest. In preferred embodiments, it is contemplated that a biodegradable matrix that is capable of being reabsorbed into the body will likely be most useful.

To improve placental derived stem cell adhesion to the matrix, and survival and function of the placental derived stem cell, the matrix may optionally be coated in its external surface with factors known in the art to promote cell adhesion, growth or survival. Such factors include cell adhesion molecules, extra cellular matrix molecules or growth factors.

The present invention also relates to the use of placental derived stem cells in three dimensional cell and tissue culture systems to form structures analogous to tissue counterparts in vivo. The resulting tissue will survive for prolonged periods of time, and perform tissue-specific functions following transplantation into the recipient host. Methods for producing such structures is described in U.S. Pat. No. 5,624,840, which is incorporated herein in its entirety. The present invention further relates to the use of the matrix/hepatic cell cultures for generation of three-dimensional hepatic cell culture systems to form structures analogous to liver tissue counterparts. Cells cultured on a three-dimensional matrix will grow in multiple layers to develop organotypic structures occurring in three dimensions such as ducts, plates, and spaces between plates that resemble sinusoidal areas, thereby forming new liver tissue. Thus, in preferred aspects, the present invention provides a three-dimensional, multi-layer cell and tissue culture system. The resulting liver tissue culture system survives for prolonged periods of time and performs liver-specific functions for use as a perfusion device or following transplantation into the recipient host.

The present methods and compositions described herein may employ placental derived stem cells genetically engineered to enable them to produce a therapeutic protein to treat a subject. The therapeutic protein can used to correct a metabolic deficiency in a subject. As used herein the term “therapeutic protein” includes a wide range of functionally active biologically active proteins including, but not limited to, growth factors, enzymes, hormones, cytokines, inhibitors of cytokines, blood clotting factors, peptide growth and differentiation factors. Pancreatic cells can be engineered to produce digestive enzymes. Hepatocytes can be engineered to produce the enzyme inhibitor, A1AT, or produce clotting factors to treat hemophilia. Furthermore, neuronal cells can be engineered to produce chemical transmitters.

Methods which are well known to those skilled in the art can be used to construct expression vectors containing a nucleic acid encoding the protein of interest linked to appropriate transcriptional/translational control signals. See, for example, the techniques described in Sambrook, et al., 1992, Molecular Cloning, A Laboratory Manual, Cold Spring Harbor Laboratory, N.Y., and Ausebel et al., 1989, Current Protocols in Molecular Biology, Greene Publishing Associates & Wiley Interscience, N.Y.

Suitable methods for transferring vector or plasmids into placental derived stem cells include lipid/DNA complexes, such as those described in U.S. Pat. Nos. 5,578,475; 5,627,175; 5,705,308; 5,744,335; 5,976,567; 6,020,202; and 6,051,429. Suitable reagents include lipofectamine, a 3:1 (w/w) liposome formulation of the poly-cationic lipid 2,3-dioleyloxy-N-[2(sperminecarbox-amido)ethyl]-N,N-dimethyl-1-propanaminium trifluoroacetate (DOSPA) (Chemical Abstracts Registry name: N-[2-(2,5-bis[(3-aminopropyl)amino]-1-oxpentyl}amino)ethyl]-N,N-dimethyl-2,3-bis(9-octadecenyloxy)-1-propanamin-iumtrifluoroacetate), and the neutral lipid dioleoyl phosphatidylethanolamine (DOPE) in membrane filtered water. Exemplary is the formulation Lipofectamine 2000™ (available from Gibco/Life Technologies # 11668019). Other reagents include: FuGENE™ 6 Transfection Reagent (a blend of lipids in non-liposomal form and other compounds in 80% ethanol, obtainable from Roche Diagnostics Corp. # 1814443); and LipoTAXI™ transfection reagent (a lipid formulation from Invitrogen Corp., produce the desired biologically active protein. #204110). Transfection of placental derived stem cells can be performed by electroporation, e.g., as described in M. L. Roach and J. D. McNeish (2002) Methods in Mol. Biol. 185:1. Suitable viral vector systems for producing stem cells with stable genetic alterations may be based on adenoviruses, lentiviruses, retroviruses and other viruses, and may be prepared using commercially available virus components.

Compositions of the present invention also include placental derived stem cells, or placental derived stem cells induced to differentiate, in a pharmaceutically acceptable carrier for administration into a recipient host in need of new tissue. Cell compositions for administration to a subject in accordance with the present invention thus may be formulated in any conventional manner using one or more physiologically acceptable carriers comprising excipients and auxiliaries which facilitate processing of the compounds into preparations which can be used pharmaceutically. Proper formulation is dependent upon the route of administration chosen. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. The compositions may be packaged with written instructions for use of the cells in tissue regeneration, or restoring a therapeutically important metabolic function. Placental derived stem cells may also be administered to the recipient in one or more physiologically acceptable carriers. Carriers for these cells may include, but are not limited to, solutions of phosphate buffered saline (PBS) or lactated Ringer's solution containing a mixture of salts in physiologic concentrations.

### 5.1. Growth and Culture of Placental Derived Stem Cells

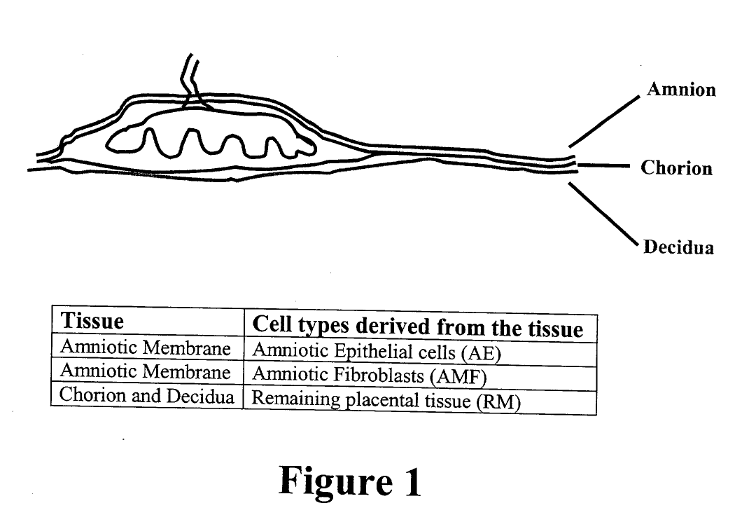

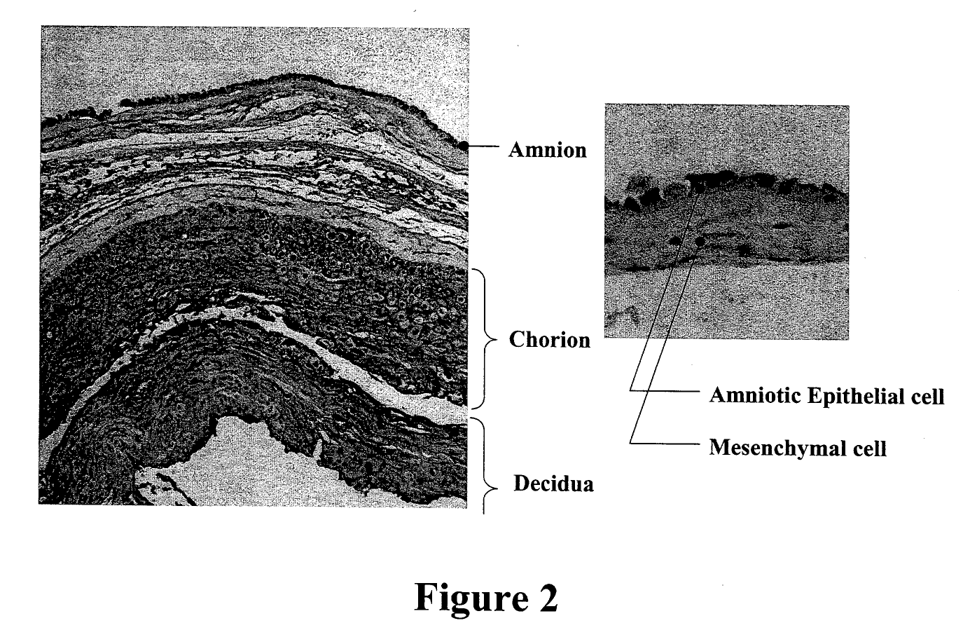

The present invention provides novel placental derived stem cells that can be obtained from the amnion, chorion or decidual layers of the placenta. In a specific embodiment of the invention, placental-derived stem cells are isolated from the amniotic membrane and associated mesenchyme (FIGS. 1 and 2). This may be readily accomplished using techniques known to those skilled in the art. For example, amniotic cells may be aspirated from amniotic fluid. Alternatively, the amniotic tissue may be dissected free of chorion and other placental tissues. The amnion layer may be gently stripped from the underlying chorion layer using forceps and a sterile scalpel. The amnion layer can be disaggregated mechanically and/or treated with digestive enzymes and/or chelating agents that weaken the connections between neighboring cells, making it possible to disperse the tissue suspension of individual cells. In yet another embodiment of the invention the chorion or decidua of the placenta can also be used as a source of placental stem cells for the present invention.

Enzymatic dissociation can be carried out by treating the amnion layer with any of a number of digestive enzymes. Such enzymes include, but are not limited to, trypsin, chymotrypsin, collagenase, elastase and/or hylauronidase. In a preferred embodiment of the invention, the isolated amniotic tissue is treated with trypsin to dissociate individual cells. In a more preferred embodiment, the concentration of trypsin for incubation of the tissue is 0.05%. In one embodiment of the invention, the tissue is subjected to digestion with enzyme for varying periods of time, preferably between 10 and 40 minutes, most preferably for 30 minutes. The tissue may also be subjected to multiple treatments with enzymes. A review of tissue disaggregation technique is provided in, e.g., Freshney, Culture of Animal Cells, A Manual of Basic Technique, 2d Ed., A. R. Liss, Inc., New York, 1987, Ch. 9, pp.107-126.

Following preparation of a single cell suspension, the cells can be cultured in medium containing a basal medium, supplemented with serum, hormones, growth factors, cytokines antibiotics, trace elements and other additives. Growth factors and cytokines may include fibroblast growth factors (FGFs), epidermal growth factor (EGF), transforming growth factor-β (TGF-β), hepatocyte growth factor (HGF) or oncostatin M. Additives to the medium may include insulin, transferrin, selenium (ITS), glucose, interleukin 6 and histone deacetylase inhibitors such as sodium butyrate or tricostatin A.

In a specific embodiment of the invention, placental-derived cells are plated onto dishes with DMEM, 10% FBS, 2 mM L-glutamine, EGF (10 ng/ml), insulin (10 μg/ml), transferrin (5.5 μg/ml), selenium (6.7 ng/ml) and ethanolamine (2 μg/ml). In addition, sodium pyruvate and non-essential amino acids (1%) may be added to the culture medium. Those of skill in the art will also recognize that one or more commercially available substances may be used as additives or substitutions to the medium to support the growth of stem cells. To induce demethylation or dedifferentiation, 5-azacytidine and/or BMP inhibitors may also be added to the medium. The cells may be cryopreserved and retain function and viability when thawed.

The cells may be plated on tissue culture dishes or may be grown in a cell suspension in a flask, forming spheroidal cell bodies. When grown on tissue culture dishes, the surface may be coated electrostatically or with extracellular matrix components. Cells may be passaged before reaching confluency on the dish to avoid contact inhibition and maintain proliferating growth conditions.

Additionally, cells can be grown by culture with placental stromal cells or co-culture with progenitor or differentiated cells derived from different organs and tissue.

In addition, the cells may be grown on feeder layers. In culturing the cells of the invention, it is believed that the use of feeder cells, or an extracellular matrix derived from feeder cells, provides one or more substances necessary to promote the growth of the stem cells and/or inhibits the rate of differentiation of such cells. Such substances are believed to include membrane-bound and/or soluble cell products that are secreted into the surrounding medium by the cells. For example, placental derived stem cells can be grown on a substrate selected from the group consisting of mouse embryo fibroblast cells, STO cells, human fibroblasts, or human epithelium cells. Thus, those of skill in the art will recognize that additional cell lines can be used with the cell culture medium to equivalent effect and that such additional cell lines can be identified using standard methods and materials. In addition, those of skill in the art will also recognize that one or more substances produced by the feeder cells, or contained in the extracellular matrix, can be identified and added to the cell culture medium of the invention to obviate the need for such feeder cells and/or such extracellular matrix.

In addition, cell surface markers such as SSEA1, SSEA3, SSEA4, TRA1-60, TRA1-81, Thy-1, and c-kit may be used to purify enriched populations of cells using a variety of methods. Such procedures involve a positive selection, such as passage of sample cells over a column containing anti-SSEA1, anti-SSEA3, anti-SSEA4, anti-TRA1-60, anti-TRA1-81, anti-Thy-1 or anti-c-kit antibodies or binding of cells to magnetic bead conjugated anti-SSEA1, anti-SSEA3, anti-SSEA4, anti-TRA1-60 anti-TRA1-81, anti-Thy-1 or anti-c-kit or by panning on anti-SSEA1, anti-SSEA3, anti-SSEA4, anti-TRA1-60, anti-TRA1-81, anti-Thy-1 or anti-c-kit antibody coated plates and collecting the bound cells. Alternatively, the single cell suspension may be exposed to a labeled antibody that immuno-specifically binds to the SSEA1, SSEA3, SSEA4, TRA1-60, TRA1-81, Thy-1 or c-kit cell surface antigen. Following incubation, with the SSEA1, SSEA3, SSEA4, TRA1-60, TRA1-81, Thy-1 or c-kit antibody, the cells are rinsed in buffer to remove any unbound antibody. Cells expressing the SSEA1, SSEA3, SSEA4, TRA1-60, TRA1-81, Thy-1 or c-kit cell surface antigen can then be cell sorted by fluorescence-activated cell sorting using, for example, a Becton Dickinson FACStar flow cytometer.

In certain embodiments, placental derived stem cells can be stably transfected with a marker that is under the control of a tissue-specific regulatory region as an example, such that during differentiation, the marker is selectively expressed in the specific cells, thereby allowing selection of the specific cells relative to the cells that do not express the marker. The marker can be, e.g., a cell surface protein or other detectable marker, or a marker that can make cells resistant to conditions in which they die in the absence of the marker, such as an antibiotic resistance gene. These methods are further described, e.g., in U.S. Pat. No. 6,015,671.

Prior to transplantation into the recipient host, the placental derived stem cells may be contacted with a number of different growth factors that can affect cell proliferation, differentiation and gene expression. Such growth factors include those capable of stimulating the proliferation and/or differentiation of stem cells, for example, but not limited to, epidermal growth factor (EGF), transforming growth factor-β (TGF-β), hepatocyte growth factor/scatter factor (HGF/SF), or fibroblast growth factors (FGFs).

### 5.2. Differentiated Hepatocytes and Methods of Use

Placental derived stem cells can be cultured to generate hepatocytes. The term “hepatocytes” as used herein refers to cells that have characteristics of epithelial cells obtained from liver, for example cells that express asialoglycoprotein receptor (ASGR), alpha-1-antitrypsin (A1AT), albumin, hepatocyte nuclear factors (HNF1 and HNF4) and CYP genes (1A1, 1A2, 2C8, 2C9, 2D6, 3A4). Other markers of interest for hepatocytes include α1-antitrypsin, glucose-6-phosphatase, transferrin, CK7, γ-glutamyl transferase; HNF 1β, HNF 3α, HNF-4α, transthyretin, CFTR, apoE, glucokinase, insulin growth factors (IGF) 1 and 2, IGF-1 receptor, insulin receptor, leptin, apoAII, apoB, apoCIII, apoCII, aldolase B, phenylalanine hydroxylase, L-type fatty acid binding protein, transferrin, retinol binding protein, erythropoietin (EPO), and clotting factors, such as Factor V, VII, VIII, IX and X.

Hepatocytes may also display the following biological activities, as evidenced by functional assays. The cells may have a positive response to dibenzylfluorescein (DBF), have the ability to metabolize certain drugs, e.g., dextromethorphan and coumarin; have drug efflux pump activities (e.g., P glycoprotein activity); upregulation of CYP activity by phenobarbital, as measured, e.g., with the pentoxyresorufin (PROD) assay, which is seen only in hepatocytes and not in other cells (see, e.g., Schwartz et al. (2002) J. Clin. Invest. 109:1291); take up LDL, e.g., Dil-acil-LDL (see, e.g., Schwartz et al., supra); store glycogen, as determined, e.g., by using a periodic acid-Schiff (PAS) staining of the cells (see, e.g., Schwartz et al., supra); produce urea and albumin (see, e.g., Schwartz et al., supra); and present evidence of glucose-6-phosphatase activity.

In a specific embodiment of the present invention, isolated placental derived stem cells are cultured in optimal differentiation media to promote differentiation into hepatocytes. Media supplemented with various growth factors, or combination of factors, can be used to promote such cell differentiation. To promote placental derived stem cell differentiation into hepatocytes, cells can be cultured in basal medium supplemented with one or more of the following growth factors, EGF (0.1-100 ng/ml), Dexamethasone (0.1-100 μM), HGF (0.1-100 ng/ml), ITS (Insulin (0.1-100 μg/ml), Transferrin (0.1-100 μg/ml), Selenium (0.1-100 ng/ml), Ethanolamine (0.1-100 μg/ml). Preferably, to obtain placental derived stem cells capable of optimal differentiation into hepatocytes, cells are cultured in 10 ng/ml EGF, 1μM Dexamethasone, 10 μg/ml Insulin, 5.5 μg/ml Transferrin, 6.7 ng/ml Selenium, and 2 μg/ml Ethanolamine.

In an additional embodiment, the invention provides enriched populations of hepatocyte-like cells. Exemplary populations of cells comprise at least about 50%; preferably at least about 60%; 70%; 80%; 90%; 95%; 98% and most preferably 99% of hepatocyte cells. Hepatocytes may be enriched by the detection of tissue-specific markers by immunological techniques, such as flow immunocytochemistry for cell-surface markers, immunohistochemistry (for example, of fixed cells or tissue sections) for intracellular or cell-surface markers, Western blot analysis of cellular extracts, and enzyme-linked immunoassay, for cellular extracts or products secreted into the medium. The expression of tissue-specific gene products can also be detected at the mRNA level by Northern blot analysis, dot-blot hybridization analysis, or by reverse transcriptase initiated polymerase chain reaction (RT-PCR) using sequence-specific primers in standard amplification methods.

Placental derived stem cells that have been differentiated into hepatocytes can be administered in the treatment of liver diseases, such as in artificial liver devices (BAL-bioartificial liver) or for hepatocyte transplant. The term “liver diseases” as used herein includes but is not limited to cirrhosis of the liver, metabolic diseases of the liver, such as alpha 1-antitrypsin deficiency and ornithine transcarbamylase (OTC), alcohol-induced hepatitis, chronic hepatitis, primary sclerosing cholangitis, alpha 1-antitrypsin deficiency and liver cancer.

The stem cells of the invention are administered to the recipient in an effective amount to achieve its intended purpose. More specifically, an effective amount means an amount sufficient to lead to the development of new tissue and restoration of tissue function, thereby alleviating the symptoms associated with disorders resulting from genetic defects or tissue damage.

The number of cells needed to achieve the purposes of the present invention will vary depending on the degree of tissue damage and the size, age and weight of the host. Determination of effective amounts is well within the capability of those skilled in the art. The effective amount may be determined by using a variety of different assays designed to detect restoration of tissue function. More specifically, assays may be used to detect the activity of specific metabolic pathways. The progress of the transplant recipient can be determined using assays that include blood tests known as liver function tests. Such liver function tests include assays for alkaline phosphates, alanine transaminase, aspartate transaminase and bilirubin circulating levels of liver derived clotting factors and determination of clotting times. In addition, recipients can be examined for presence or disappearance of features normally associated with liver disease such as, for example, jaundice, anemia, leukopenia, thrombocytopenia, increased heart rate, and high levels of insulin. Additionally, assays specific for measuring deficiencies in particular metabolic disorders may also be used. Further, imaging tests such as ultrasound, computer assisted tomography (CAT) and magnetic resonance (MR) may be used to assay for liver function.

In addition, the cultures of cells are propagated in the presence of a natural or synthetic matrix that provides support for hepatic cell growth during in vitro culturing. The type of matrix that may be used in the practice of the invention is virtually limitlessness. The matrix will have all the features commonly associated with being “biocompatible”, in that it is in a form that does not produce an adverse, or allergic reaction when administered to the recipient host. For purposes of forming bio-artificial livers, the matrix may be composed of any suitable material to which the hepatocytes and nonparenchymal cells will adhere and proliferate.

Further, to improve hepatic cell adhesion, proliferation or survival, the matrix may be coated on its external surface with factors known in the art to promote cell adhesion, growth or survival. Such factors include cell adhesion molecules, extra-cellular matrix molecules and/or growth factors for hepatocytes. Matrices may also be designed to allow for sustained release of growth factors over prolonged periods of time. Thus, appropriate matrices will ideally provide factors known to promote hepatic cell adhesion, growth or survival, and also act as a support on which the cultured cells differentiate and proliferate.

The conditions of long-term matrix-cell culturing will preferably be maximized to enhance hepatocyte proliferation while maintaining hepatic function. Although certain variations in cell number, seeding techniques, culture media, incubation temperatures and incubation times, may be utilized, such variations would be routine to those skilled in the art and are encompassed by the present invention.

The present invention further relates to the use of the matrix/hepatic cell cultures, for generation of three-dimensional hepatic cell culture systems to form structures analogous to liver tissue counterparts. The method of the invention comprises growing hepatic cells on a three-dimensional matrix in vitro under conditions effective and for a period of time sufficient to allow proliferation of the cells to form a three-dimensional structure.

The three-dimensional matrices to be used are structural matrices that provide a scaffold for the cells, to guide the process of tissue formation. Cells cultured on a three-dimensional matrix will grow in multiple layers to develop organotypic structures occurring in three dimensions such as ducts, plates, and spaces between plates that resemble sinusoidal areas, thereby forming new liver tissue. Thus, in preferred aspects, the present invention provides a three-dimensional, multi-layer cell and tissue culture system.

To form the bio-artificial liver the three-dimensional hepatocyte cell cultures of the invention are grown within a containment vessel containing an input and output outlet for passage of the subjects blood through the containment vessel. The bio-artificial liver further includes a blood input line which is operatively coupled to a conventional peristaltic pump. A blood output line is also included. Input and output lines are connected to appropriate arterial-venous fistulas which are implanted into, for example, the forearm of a subject. In addition, the containment vessel may contain input and output outlets for circulation of appropriate growth medium to the hepatocytes for continuous cell culture within the containment vessel.

The use of such bio-artificial livers involves the perfusion of the subject's plasma through the bio-artificial liver. In the perfusion protocol, the subject's blood or plasma is withdrawn and passes into contact with the hepatocyte cell cultures. During such passage, molecules dissolved in the patient's blood, such as bilirubin, are taken up and metabolized by the hepatocyte cultures. In addition, the cultured hepatocytes provide factors normally supplied by liver tissue.

The hepatocytes of the invention may be administered in a manner that permits them to graft to the intended tissue site and reconstitute or regenerate the functionally deficient area. Hepatocytes can be used in therapy by direct administration, or as part of a bioassist device that provides temporary liver function while the subject's liver tissue regenerates itself following fulminant hepatic failure.

Hepatocytes of the invention can be assessed in animal models for ability to repair liver damage. One such example is damage caused by intraperitoneal injection of D-galactosamine (Dabeva et al., Am. J. Pathol. 143:1606, 1993). Efficacy of treatment can be determined by immunocytochemical staining for liver cell markers, microscopic determination of whether canalicular structures form in growing tissue, and the ability of the treatment to restore synthesis of liver-specific proteins.

In another embodiment, the differentiated hepatocytes may be used for testing whether test agents such as lead drug compounds have a negative biological effect on hepatocytes. For example, the hepatocyte cell preparation is incubated in the presence or absence of a test compound for a time sufficient to determine whether the compound may be cytotoxic to cells. Cells can be incubated with various concentrations of a test compound. In an illustrative embodiment, cells are plated in the wells of a multi-well plate to which different concentrations of the test compound are added, e.g., 0 μM; 0.01 μM; 0.1 μM; 1 μM; 10 μM; 100 μM; 1 mM; 10 mM and 100 mM. Cells can be incubated for various times, e.g., 1 minute, 10 minutes, 1 hour, 2 hours, 5 hours, 10 hours, 24 hours, 36 hours or more.

The biological effect that is measured can be triggering of cell death (i.e., cytotoxicity or hepatotoxicity); a cytostatic effect; or a transforming effect on the cell, as determined, e.g., by an effect on the genotype or phenotype of the cells. The cytotoxicity on cells can be determined, e.g., by incubating the cells with a vital stain, such as trypan blue. Such screening assays can easily be adapted to high throughput screening assays.

Hepatocytes derived from differentiated placental derived stem cells of the invention can also be used for metabolic profiling. In one embodiment, cells or a fraction thereof, e.g., a microsome fraction, are contacted with a test agent, potentially at different concentrations and for different times, the media is collected and analyzed to detect metabolized forms of the test agent. Optionally, a control molecule, such as bufuralol is also used. Metabolic profiling can be used, e.g., to determine whether a subject metabolizes a particular drug and if so, how the drug is metabolized.

### 5.3. Differentiated Pancreatic Cells and Methods of Use

In a specific embodiment of the invention, the present invention also provides for methods of differentiating placental derived stem cells into pancreatic-like cells. As used herein, the term “pancreatic-like cells” is comprised of four types of cells. The α cells constitute about 20% of the cells found in pancreatic islets and produce the hormone glucagon. Glucagon acts on several tissues to make energy available in the intervals between feeding. In the liver, glucagon causes breakdown of glycogen and promotes gluconeogenesis from amino acid precursors. The δ cells produce somatostatin which acts in the pancreas to inhibit glucagon release and to decrease pancreatic exocrine secretion. The hormone pancreatic polypeptide (PP) is produced in the PP cells. This hormone inhibits pancreatic exocrine secretion of bicarbonate and enzymes, causes relaxation of the gallbladder, and decreases bile secretion. The most abundant cell in the islets, constituting 60-80% of the cells, is the 13 cell, which produces insulin. Insulin is known to cause the storage of excess nutrients arising during and shortly after feeding. The major target organs for insulin are the liver, muscle, and fat-organs specialized for storage of energy. The differentiated pancreatic islets cells may be positive for markers such as Nkx-2.2, glucagon, Pax6, Pdx1, and insulin.

In an embodiment of the invention, isolated placental cells may be cultured in optimal differentiation media to promote pancreatic cell differentiation. Cells may be maintained in a standard growth media for approximately 7 days followed by trypsinization and seeding on culture substrates coated with Matrigel™ (MG). Culture substrates may be coated with 20% to 100% (v/v MG to media) MG. Cells are seeded in plates previously coated with MG and cultured an additional 14 days in standard media supplemented with dexamethasone (0.1 μm) and ITS.

The present invention also provides for pancreatic cells derived from placental derived stem cells which can be used therapeutically for treatment of various diseases associated with insufficient functioning of the pancreas. As used herein, the term “pancreatic diseases” may include but is not limited to pancreatic cancer, insulin-deficiency disorder such as Insulin-dependent (Type 1) diabetes mellitus (IDDM) and Non-insulin-dependent (Type 2) diabetes mellitus (NIDDM), hepatitis C infection, exocrine and endocrine pancreatic diseases.

Example 14 shows the results of cultured placental derived stem cells that express pancreatic islet cell markers, in particular, insulin. The expression of insulin in these cultured cells indicate that they have the potential to be used therapeutically. These cells may secrete or be induced to secrete insulin for use towards the treatment of diabetes.

The placental derived stem cells can be used to produce populations of differentiated pancreatic cells for repair subsequent to partial pancreatectomy, e.g., excision of a portion of the pancreas. Likewise, such cell populations can be used to regenerate or replace pancreatic tissue loss due to, pancreatolysis, e.g., destruction of pancreatic tissue, such as pancreatitis, e.g., a condition due to autolysis of pancreatic tissue caused by escape of enzymes into the substance. Pancreatic cells may be transplanted into the pancreas or to ectopic sites, such as, but not limited to the liver, kidney or at or near the intestines.

Methods of administration include encapsulating differentiated β islet cells producing insulin in implantable hollow fibers. Such fibers can be pre-spun and subsequently loaded with the differentiated β islet cells of the invention (Aebischer et al. U.S. Pat. No. 4,892,538; Aebischer et al. U.S. Pat. No. 5,106,627; Hoffman et al. (1990) Expt. Neurobiol. 110:39-44; Jaeger et al. (1990) Prog. Brain Res. 82:41-46; and Aebischer et al. (1991) J. Biomech. Eng. 113:178-183), or can be co-extruded with a polymer which acts to form a polymeric coat about the B islet cells (Lim U.S. Pat. No. 4,391,909; Sefton U.S. Pat. No. 4,353,888; Sugamori et al. (1989) Trans. Am. Artif. Intern. Organs 35:791-799; Sefton et al. (1987) Biotehnol. Bioeng. 29:1135-1143; and Aebischer et al. (1991) Biomaterials 12:50-55).

### 5.4. Differentiated Cells of the Nervous Tissue and Methods of Use

In an exemplary embodiment of the invention, isolated placental derived stem cells are cultured in optimal differentiation media to promote differentiation into cells of the nervous tissue. As used herein the term “nervous tissue” may include but are not limited to cells from central and peripheral nervous tissue that contain neurons, glial cells, oligodendrocytes, and astrocytes. Such cells may be characterized by the presence of markers such as GFAP, beta-tubulin, CNP, or FLT1.

The present invention also provides for administration of nervous tissue cells derived from placental derived stem cells for treatment of various neurological diseases. The term “neurological disease” refers to a disease or condition associated with any defects in the entire integrated system of nerve tissue in the body: the brain, brainstem, spinal cord, nerves and ganglia. Examples include but are not limited to: Parkinson's disease, Huntington's disease, choreic syndrome, dystonic syndrome, and paralysis.

Cells can be cultured in basal medium supplemented with one or more of the following growth factors, EGF (0.1-100 ng/ml), Dexamethasone (0.1-100 μM), HGF (0.1-100 ng/ml), ITS (Insulin (0.1-100 μg/ml), Transferrin (0.1-100 μg/ml), Selenium (0.1-100 ng/ml) Ethanolamine (0.1-100 μg/ml) and, in particular, with FGF-4, preferably in the range of 10 ng/ml.

### 5.5. Differentiated Vascular Endothelial Cells and Methods of Use

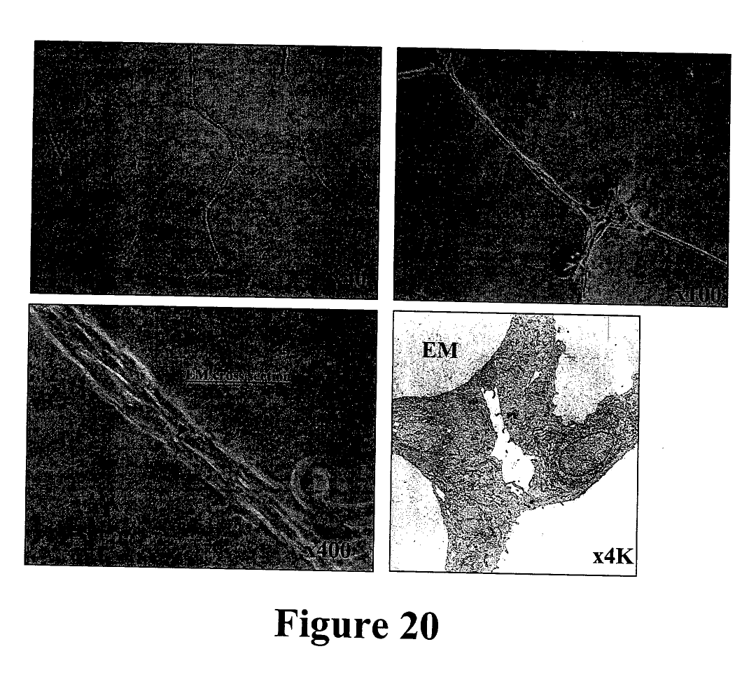

In certain embodiments, the invention relates to methods for culturing placental derived stem cells including but not limited to generating vascular endothelial cells. In another embodiment, the invention provides for methods wherein the placental derived stem cells are cultured in a matrigel containing media under appropriate conditions and for a sufficient period of time to induce vascular endothelial cell differentiation. As used herein, the term “vascular endothelial cells” refers to endothelial cells, which line the interior of blood vessels and have essential physiological functions that include modulation of vasoreactivity and provision of a semi-permeable barrier to plasma fluid and protein. Vascular endothelial cells can be characterized as follows. They may have a phenotype similar to that of the cells shown in FIG. 20. In an exemplary embodiment, a pharmaceutical composition comprising the vascular endothelial cells derived from placental stem cells in an effective amount, may be used to treat a subject with a vascular disease. As used herein, “vascular disease” refers to a disease of the human vascular system. Examples include peripheral arterial disease, abdominal aortic aneurysm, carotid disease, venous disease.

### 5.6. Cryopreservation of Placental-Derived Cells

Placental stem cells may be cryopreserved and thawed with no discernable loss of function. Placental-derived cells were isolated from the amnion as described and cultured in basal media for 7-10 days or until the cultures grew to confluence. Cells were trypsinized, washed 1× to remove trypsin and counted. Placental stem cells were cryopreserved by suspending the isolated cells in basal media (90%) supplemented with Dimethylsulfoxide (DMSO) (10% v/v). Cells were cryopreserved by placing them in a cell freezer container which when placed into a −80 degree C. freezer to cool the cells at a rate of approximately one degree C. per minute. Cells were stored at −80 C. until needed. Cells were thawed rapidly by placing the vials in a water bath pre-warmed to 37 degrees C. Upon complete thawing cell were decanted from the cryovials and added to at least 3 volumes of pre-warmed (37 degrees C.) basal media. Cells were centrifuged at 100×g for 5 minutes. Cells were resuspended in basal media counted and checked for viability and plated on regular culture dishes. Viabilities of the thawed cells ranged from 70-95% in freezes of different batches of placental-derived cells. This is a standard cryopreservation technique used by many cell culturists. Glycerol may be used in place of DMSO at a concentration ranging from 5-40%, DMSO may be used at concentrations ranging from 5-35%, and different media may be substituted for the basal media used here. Different media could include but are not limited to balanced salts solution such as HBSS, any complete tissue culture media such as MEM, DMEM, F12, etc. Cryopreservation solutions may consist of a solution used for the cold storage and transportation of organs from transplantation such as Belzer's UW solution or HKT or and equivalent. The cryopreservation rate of approximately 1 degree per minute is a standard rate but the cryopreservation results may be improved by using different rates allowable through the use of a programmable cell freezer. Cells recovered from cryopreservation attach to culture plated and grow at a rate not discernibly different from cells not previously frozen.

### 6. EXAMPLE

### Placental Tissues Contain Cells with Markers of Totipotent Stem Cells

### 6.1. Materials and Methods

### 6.1.1 isolation of Placental-Derived Cells from Different Section of Placenta

The populations of placental-derived cells are isolated from various sections of the placenta. Placental-derived cells are isolated from the amniotic membrane which is easily peeled off of the placental body (FIG. 1) and contains the amniotic epithelial cells and a supportive stromal layer (FIG. 2). The stromal layer contains mesenchymal cells, or fibroblastic cells as well as other cell types. The amniotic membrane was peeled off of the placenta and was trypsinized to release amniotic epithelial cells. Cells which are derived from the tissue which remains following trypsinization are labeled amniotic fibroblasts (AMF). At this point in the research this fraction is more operationally defined by the mechanism by which cells are released and the tissue from which the cells are derived rather than by histochemically defined cell types. Although the exact cell types in this stromal layer are not fully characterized and defined, to simplify the wording and for the purposes of this application we will call them amniotic fibroblasts (AMF) with full understanding that cell types other than fibroblasts are most likely contained in what we call the AMF fraction.

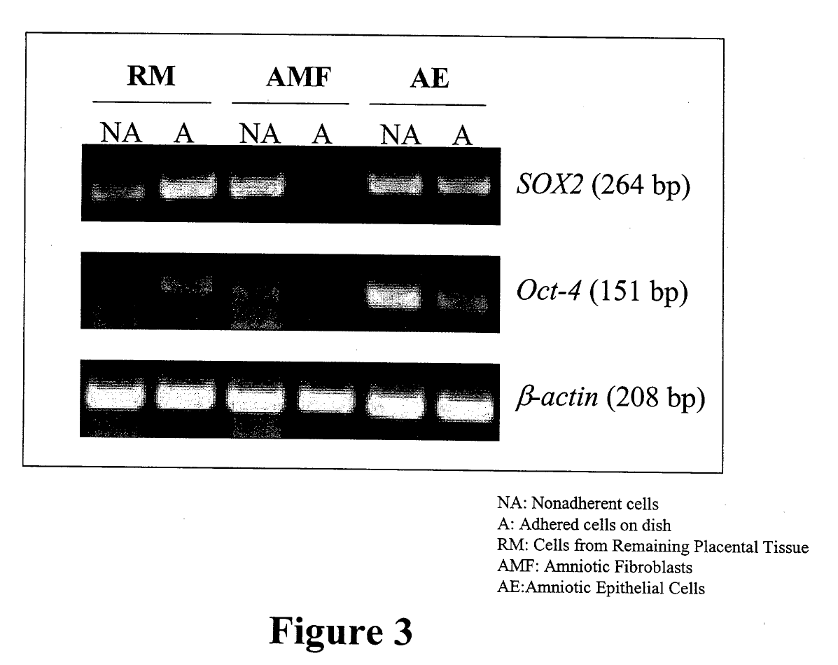

The amniotic membrane was peeled from the placenta and was trypsinized to release the amniotic epithelial cells. Cells derived from tissue which remains following initial trypsinization are labeled as amniotic fibroblasts (AMF). The amnion layer was peeled off and the remaining placental membrane was digested with collagenase. The cells derived from the remaining tissue was labeled RM. Cells of each fraction (AE, AMF, RM) were plated on plastic culture dishes in basal plating media. At 20 hrs following plating, the cultures were examined. Some cells were attached to the culture dish, referred to as the adherent fraction (FIG. 3). The remaining cells which did not adhere to the plastic were collected and are represented in FIG. 3 as non-adherent (NA). As stem cells in certain tissues seem to reside in the nonadherent fractions, it is significant that cells with stem cell markers can be found in each of these fractions from placenta. It is not known whether the cells with stem cell characteristics from each fraction are identical. Total cellular RNA was collected from the adherent and non-adherent cells from each of the placental fractions.

Total RNA was extracted with RNAWIZ (Ambion). RT-PCR was performed with Super Script One-step RT-PCR system (GIBCO, 10928-018) with a human albumin specific primers that were designed to span two-separated exons. RT-PCR with β-actin specific primers was also performed as an internal control.

### 6.1.2. Facs Analysis

Antibodies to the different antigens were incubated with isolated placental-derived cells and the resultant cell suspensions were analyzed on a flow cytometry analyzer, Beckman-Coulter Epics XL cytometer. Additional cells were analyzed for background fluorescence by incubation with a mouse IgG at the same concentration in the incubation as the highest concentration of antibody used in these studies.

### 6.2. Results

The observation that placental-derived cells contain subpopulations of cells that express Oct-4, GFAP, and FLT1 suggested that the placental-derived cells were multipotent, having the capacity to differentiate along several lineages. These results suggest that there might be ES-like cells in the placenta. These cells could give rise to all of the cell-types and tissues of the body. ES cells at different times in their growth and differentiation express what are called stage specific embryonic antigens. Antibodies to these proteins identify at least 2 different antigens, called stage specific embryonic antigens (SSEA1-4) and additional antigens called TRA 1-60 and TRA1-81. These antigens are commonly found on ES stem cells. Placental-derived cells were examined for the expression of SSEA 1-4 and the TRA antigens by FACS analysis.

The expression of Oct-4, SOX2 and hTERT in the placental-derived cells was also examined by RT-PCR analysis. Since neural stem cells express SOX2, the placental-derived cells were also evaluated for SOX2 expression. The complete results are listed in Table 1. The data in Table 1 presents results for two experiments performed.

TABLE 1

| Molecular analysis | | Immunological analysis | |
|---|---|---|---|
| -RT-PCR- | | -FCM- | |
| Oct-4 | +/+ | SSEA-1 | 1-2% |
| SOX2 | +/+ | SSEA-3 | 27% |
| hTERT | +/− | SSEA-4 | 24-94% |
| | | TRA 1-60 | 2-10% |
| | | TRA 1-81 | 1-10% |

Negative controls 0.68%

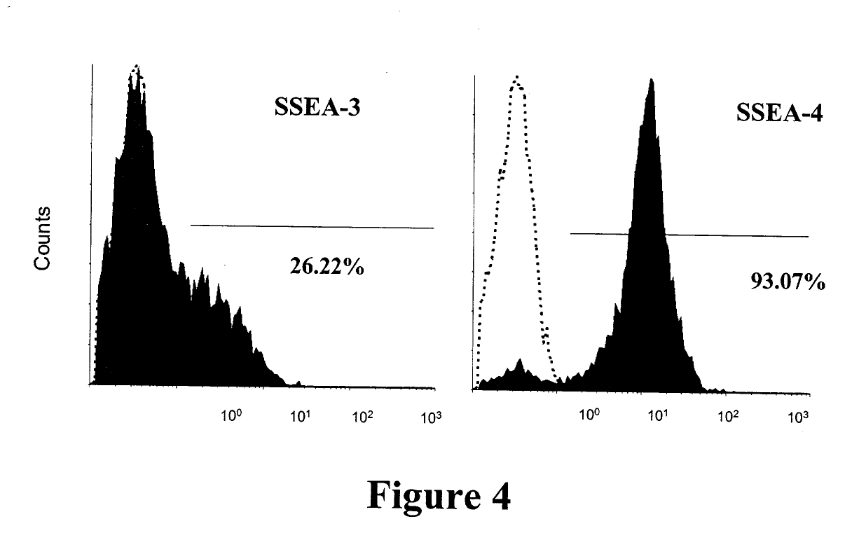

FIG. 4 shows the FACS analysis of the expression of SSEA-3 and 4 in the AE-derived cells. As indicated in Table 1, the AE-derived cells also express Oct-4 and SOX2. These results indicate that the AE-derived cells express SSEA 1, 3, and 4 and TRA1-60 and TRA1-81.

In addition, placental-derived cells derived from various portions of the placenta were analyzed for expression of Oct. 4, SOX2 and hTERT by RT-PCR as shown in Table 1. It is thought that only totipotent stem cells such as ES cells express Oct-4. The expression of Oct-4 on the placental-derived cells suggests that they are totipotent or ultimate stem cells, which could function like ES cells and give rise to all of the cell and tissue types in the body. Additional supportive evidence of ES-like totipotent stem cells is the low but detectable expression of telomerase in the placental-derived cells. Telomerase expression is also a characteristic of ES-like stem cells.

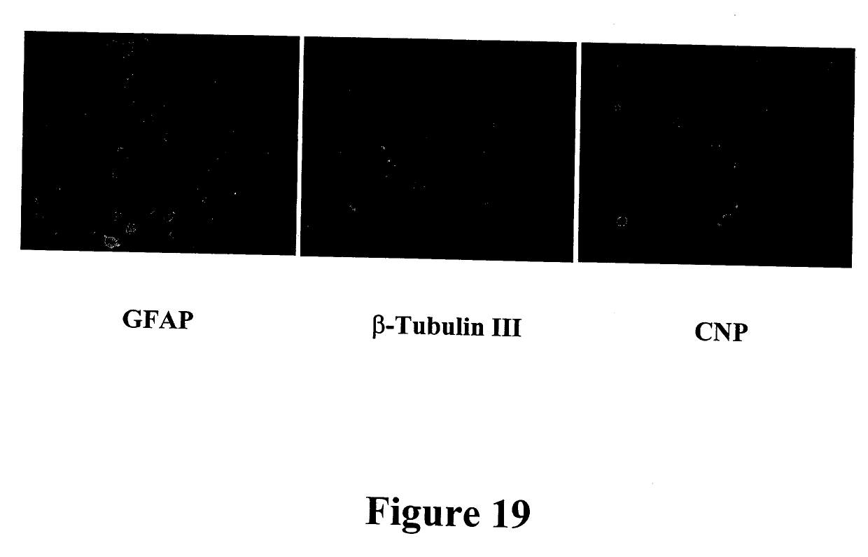

Both adherent and non-adherent fractions of the amniotic epithelial cells, the non-adherent fraction of the amniotic fibroblasts (AMF), and adherent fraction of remaining membrane (RM) contain cells that express both SOX2 and Oct-4 (FIG. 3). Amniotic epithelial (AE) cells from both fractions strongly express SOX2 and Oct-4, while other fractions express primarily SOX2 (FIG. 3). These different expression patterns of two independent stem cell marker genes indicate that different types of stem cell are capable of isolation from those fractions. Since neuro-stem cells express SOX2, the results here suggest that the amniotic epithelial fraction as well as the amniotic fibroblast fractions of the placenta contain neuro-stem cells. These cells may be useful for the neurological research and clinical transplantation for such diseases as Parkinson's or Alzheimer's or ALS, as well as other diseases of brain. Evidence has been presented in FIG. 19 of the differentiation of the placental-derived cells to different CNS cell types, confirming the biological effectiveness of the cell isolation and culture procedures described here.

Results presented in FIGS. 2, 4 and 6 and Table 1 show data, indicating that placental tissue and the amniotic membrane, in particular, contain cells which have the properties of totipotent stem cells. The cells express markers normally associated with ES-like cells such as Oct-4, hTERT, SSEA1,3 and 4 and TRA1-60 and TRA 1-81. These results indicate that ES-like cells exist in the fetal side of the placental tissue and can be easily isolated, cultured and identified. Molecular analyses of cells in culture for different times under our culture conditions indicate the continued presence of cells with ES-like characteristics. AE-derived stem cells have the potential to differentiate into all tissues and cell types of the body.

### 7. EXAMPLE

### Human Placental-Derived Cells Isolation

A human placenta was obtained from an uncomplicated elective caesarean section. The whole placenta was placed in a sterilized 1000 ml cup and washed with Hanks's Balances Salt Solution (HBSS) containing penicillin G (100 U/ml), streptomycin (100 μg/ml), and amphotericin B (0.25 μg/ml). The umbilical cord was cut and the whole placenta was cut in half at the point of attachment of the umbilical cord. The amnion layer was peeled from the underlying chorion layer of the placenta by gentle stripping with a sterile scalpel, starting from the cut edge (middle of the placental body) and working outward. The amnion was washed with HBSS (without antibiotics) and rinsed with 0.05% Trypsin-EDTA. 0.05% Trypsin-EDTA was added to approximately twice the volume of the tissue in a 50 cc Falcon tube and incubated at 37° C. for 20 min on shaker in a 5% CO₂ incubator. The tissue is transferred to a new tube with 0.05% Trypsin-EDTA. Media was added to remaining supernatant in the tube to stop trypsinization and centrifuged at 800 rpm for 10 min at 4 ° C. The pellet was resuspended in DMEM, 10% FBS, 1 mM Sodium Pyruvate, EGF (10 ng/ml), penicillin G (100 U/ml), streptomycin (100 μg/ml), and amphotericin B (0.25 μg/ml). The trysinization step was repeated up to a total of 3 times. The cells released by each trypsinization were plated separately or mixed in one tube after passing through a 100 μm cell strainer. Cells were plated onto dishes with DMEM, 10% FBS, 2mM L-glutamine, EGF (10 ng/ml), Insulin (10 μg/ml)—Transferrin (5.5 μg/ml)—Selenium (6.7 ng/ml)—Ethanolamine (2 μg/ml) (ITS). The media was changed when the cells adhere on the bottom, approximately 2-4 hrs. Media was changed every two days and the cells were passed (1 in 4) every 5 days or when the cultures reach greater than 80% confluence. Approximately 0.5-2×10⁸ cells (referred to herein as amniotic epithelial cells, i.e., AE cells) are obtainable from each placenta.

### 8. EXAMPLE

### Expression of Stem Cell, Epithelial Cell and Hepatocyte Markers in Cultured Placental-Derived Cells

### 8.1. Materials and Methods

### 8.1.1. Immunohistochemical Analysis of Placental-Derived Cells

Amniotic tissue was obtained from a normal placenta and dissociated with trypsin as described above in Example 7. Standard culture media (DMEM) was supplemented with 10% FBS, ITS and EGF (10 ng/ml). At approximately day 5 after isolation placental-derived cells were trypsinized and replated on collagen-coated cover slips, inserted into 12 well culture plates, and cultured for 2-5 days, then washed 2× with HBSS and fixed with 10% buffered formalin.

Prior to cell isolation, a small amount of tissue, approximately 1 cm×1 cm was cut from the placental tissue and fixed in 10% buffered formalin, embedded in paraffin and sectioned.

Paraffin-embedded placental tissues, sectioned to 5 μm thickness, and placental-derived cells, cultured on collagen-coated glass cover slips, were fixed by 10% buffered formalin for immunohistochemical analysis with primary antibodies against AE1/AE3, CK19, CK18, c-kit, Thy-1, A1AT, AFP. Antibody localization was performed using goat anti-mouse immunoglobulins conjugated to biotin. An avidin-biotin peroxidase complex method using DAB as a substrate (Vector) was used to develop the brown˜orange color on positive samples. A hematoxylin counter stain was performed.

Immunohistochemical analysis for human HNF-4a was prepared with rabbit anti-human HNF-4a (H171/1:250) antibody (Santa Cruz, sc-8987).

### 8.1.2. Alkaline Phosphatase Activity

Alkaline phosphatase activity was determined by Vecter Red Alkaline phosphatase substrate kit (Vector, SK-5100) (4-a:x100, 4-b:x400). Placental-derived cells were washed three times with HBSS and fixed by buffered 10% formalin for 2 hr. The red color indicative of alkaline phosphatase positivity was developed per manufacturer's instructions with a 45 min incubation at 37° C.

### 8.1.3. RT-PCR

Total RNA was extracted with RNAWIZ (Ambion). RT-PCR was performed with Super Script One-step RT-PCR system (GIBCO, 10928-018) with a human albumin specific primers that were designed to span two-separated exons. RT-PCR with β-actin specific primers was also performed as an internal control. Total RNA extracted from HeLa cells was used as negative control, and RNA from cultured human hepatocytes was used as a positive control.

### 8.1.4. Western Blotting

Placental-derived cells were homogenized in 200 μl RIPA buffer (1% TritonX-100, 150 mM NaCl, 10 mM Tris pH 7.4, 1 mM EDTA, 1 mM EGTA, 0.2 mM sodium vanadate, 0.5% NP-40) and the sample was subjected to electrophoresis on a 10% pre-cast polyacrylamide-SDS gel (Bio-Rad) at 200 V for 30 min, electrically transferred to a nitrocellulose membrane and incubated overnight at 4° C. with mouse anti-human albumin and anti-A1AT antibody.

### 8.2. Results

FIG. 7 shows the immunohistochemical analysis on placental tissue and cultured placental-derived cells. Photographs in the left column show sections through the placental tissue with a magnified insert showing the staining on the amniotic epithelial cell layer. Photos presented in the right column show the results with the isolated cultured placental-derived cells.

Both the amniotic tissue, the small single row of cells located on the upper side of the placental tissue and the isolated and cultured cells reacted strongly with antibodies to a mixture of cytokeratins (AE1/AE3) and antibodies to cytokeratins 18 and 19, indicating that the placental-derived cells are epithelial. Expression of cytokeratins 8 and 18 are markers of cells of hepatocyte lineage. Cytokeratin 19 expression in liver cells is characteristic of a biliary lineage.

Cultured human placental-derived cells reacted strongly with anti-c-kit antibodies, suggesting that these cells express this growth factor receptor. c-kit, the receptor for the hematopoietic growth factor, stem cell factor (SCF), is expressed by hematopoietic and liver stem cells.

While the amniotic tissue was negative for Thy-1 expression, cultured placental-derived cells expressed this antigen. Hematopoietic stem cells and rat liver progenitor cells express the Thy-1 antigen (Petersen B. E, Bowen W. C, Patrene K. D, Mars W. M, Sullivan A. K, Murase N, Boggs S. S, Greenberger J. S, Goff J. P: Bone Marrow As A Potential Source Of Hepatic Oval Cells. Science 1999, 284:1168-1170; Petersen et al., Hepatic Oval Cells Express The Hematopoietic Stem Cell Maker Thy-1 In The Rat. Hepatology 1998, 27:433-445). Expression of Thy-1 in placental-derived cells indicates that these cells may differentiate to cells of either hematopoietic or hepatic lineage. The cultured cells are also negative for CD34 expression.

The cultured placental-derived cells reacted with the antibody to A1AT, while the amniotic tissue was very weak or negative for A1AT expression. Alpha-1-antitrypsin (A1AT) is a protein expressed and secreted by mature hepatocytes and is a marker of hepatocyte differentiation.

The amniotic tissue is negative for AFP expression, while the cultured cells are very weakly positive for AFP expression. Alpha-fetoprotein (AFP) is the fetal form of albumin and is expressed by fetal hepatocytes before they mature. These results contrast with the report of Sakuragawa et al. wherein cultured cells were shown to express AFP (Sakuragawa et al., Human Amniotic Epithelial Cells are Promising Transgene Carriers for Allogeneic Cell Transplantation Into Liver. J Hum Genet 45:171-176, 2000).

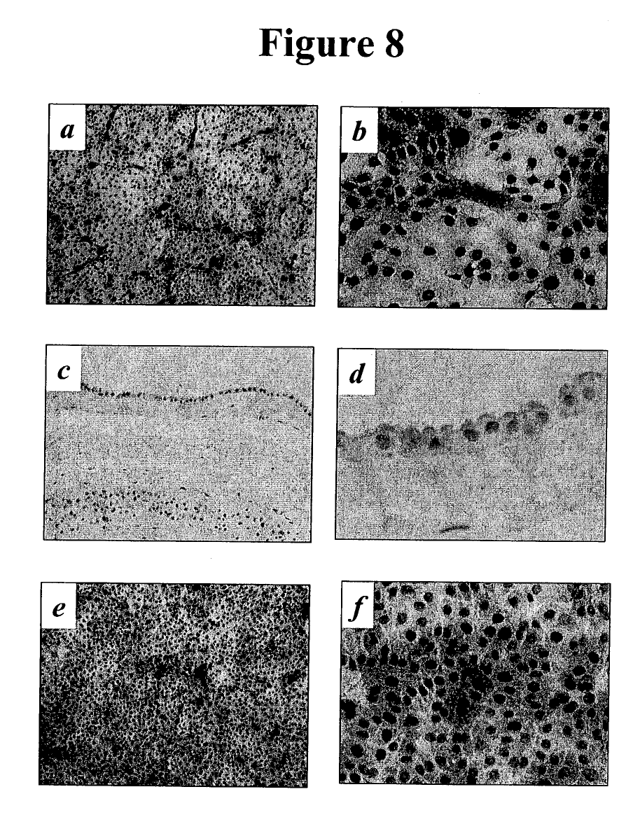

A subpopulation of the cultured cells also stained strongly for alkaline phosphatase activity. The results indicate that there are two populations of cultured cells with respect to alkaline phosphatase expression, those that strongly expressed alkaline phosphatase (FIG. 8a and b) and those demonstrated weak or negative expression (not shown). Alkaline phosphatase is a marker of undifferentiated totipotent Embryonic Stem cells (ES). When human ES cells differentiate to form embryoid bodies, expression of alkaline phosphatase is reduced or lost. This result suggests that differentiation of placental-derived cells in culture, like ES cells, is accompanied by alterations in the expression of alkaline phosphatase.

Cultured cells were also observed to express albumin, a marker of hepatocyte differentiation (FIGS. 8C-F). Localization of albumin in the population clearly indicates that there are cells which are strongly positive for albumin expression next to cells which are completely negative. Up to 30% of the cultured placental derived cells expressed albumin. It is interesting that some of the strongly albumin positive cells were binucleated, a characteristic of mature hepatocytes.

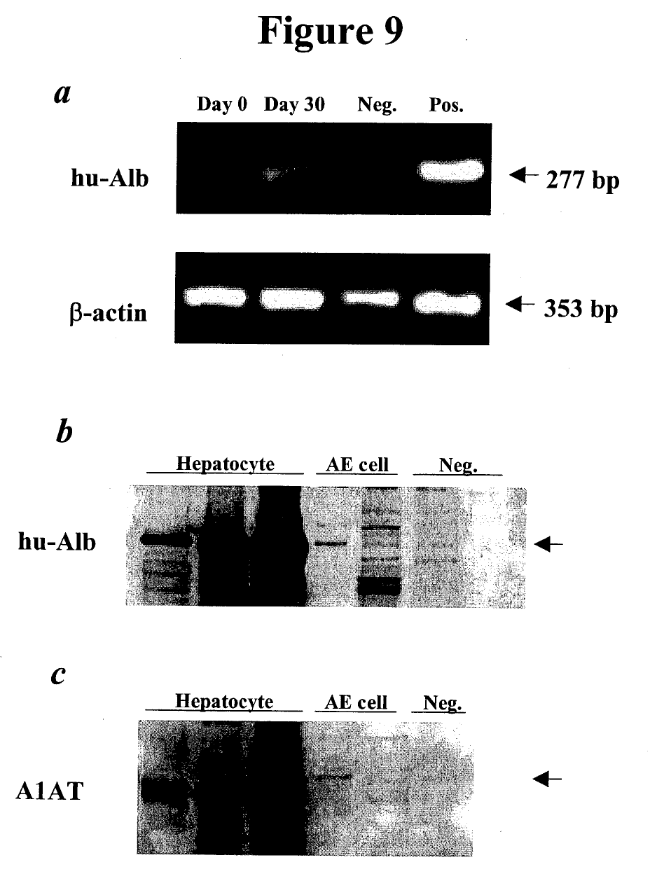

To confirm the expression of albumin at the RNA and protein level. RT-PCR and Western blot analysis were performed using cultured cells. Human albumin RNA was detected using RT-PCR from RNA isolated from cultured cells (FIG. 9A) and human albumin protein was detected using Western blot on cell extracts from cultured cells (FIG. 9B).

Alpha-1 antitrypsin, a benchmark measure of mature hepatocytes, was also detected in cell extracts from cultured cells using Western blot analysis. Cell extracts prepared from amniotic tissue, however, did not react with antibodies to albumin or A1AT, suggesting that amniotic tissue does not express albumin or A1AT in vivo. These results suggested that cells cultured under the conditions specified above proliferate and differentiate along the hepatic lineage. These data indicated that cultured placental-derived cells are not “locked” into a differentiated state in vivo, but rather, that gene expression in these cells are of a plastic nature.

TABLE 2
Expression of various markers in cultured placental-derived cells and
human placental tissue

| | | |
|---|---|---|
| Epithelial | AE1/AE3 | Cytokeratins, expressed in |
| Markers | | liver |
| | CK 18 | Cytokeratin, expressed in liver |
| | CK 19 | Cytokeratin expressed in liver |
| | | biliary and liver stem cells |
| Hepatocyte | A1AT | Alpha-1 antitrypsin |
| Markers | Alb | Albumin |
| | AFP | Alpha fetoprotein, liver |
| | | progenitor cells |
| | CYP450 genes | Drug metabolizing enzymes |
| | CYP1A1, 1A2, 2B6, 2C8, | expressed in differentiated |
| | 2C9, 2D6, 3A4 | liver |
| Stem Cell | c-kit, Thy-1, SSEA1,3,4, | expressed on stem cells |
| Markers | TRA1-60, TRA1-81, Oct-4, | |
| | SOX2, | |

There are master switches which control the pathways through which cells differentiate. Among the most important steps which regulate gene expression along certain lineages are the expression of tissue enriched transcription factors. In liver development, the expression of the Hepatocyte Nuclear Factors (HNF) are such genes. Along the hepatocytic lineage the expression of liver specific genes such as albumin are controlled by HNFs binding to the albumin promoter. In hepatocellular carcinoma cell lines which lack the expression of HNF1 and HNF3, there is no evidence of hepatocyte differentiation. However, transfection of HNF4 activates HNF1 expression and liver specific gene expression (Spath and Weiss, Hepatocyte nuclear factor expression overcomes repression of the hepatic phenotype in dedifferentiated hepatoma cells. Mol. Cell Biol 17: 1913-1922, 1997).

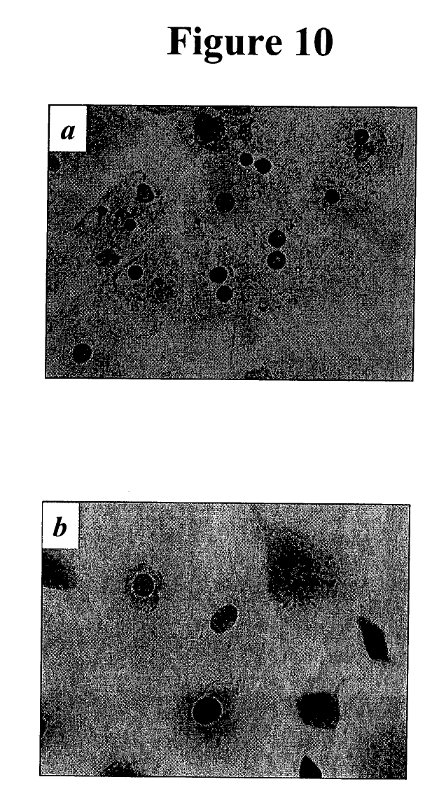

Expression of HNF 1 and 4 in cultured placental-derived cells was analyzed using immunohistochemical analysis. HNF4 localized to the nucleus in both human hepatocytes and in the cultured cells (FIG. 10). Approximately 25% of the cells exhibited detectable HNF4. Similar results were obtained with HNF1. This relative proportion of cells correlated with the proportion of albumin positive cells described above. These results also provided strong support for the plasticity of cultured placental-derived cells, i.e. that these cells can express the transcription factors and the genes required for full hepatic function.

HNF4 expression is not restricted to the liver. HNF4 expression is critical to development and differentiation in the gut, kidney, intestines and pancreatic islets. HNF4 is an important regulator of differentiation in pancreatic beta cells. In the pancreas as well as the liver, the HNF3 family of transcription factors regulate the expression of HNF4. Insulin can increase the expression of HNF3-beta leading to increased expression of HNF4 and several other genes involved in glucose metabolism. Mutations in HNF4 can lead to early onset, type 2 diabetes. In addition to the liver, HNF4 expression is critical to the normal development of the pancreatic beta cells. The observations that the cultured placental-derived cells express H4 indicates that the cultured cells may also have the ability to differentiate into insulin producing beta cells.

### 9. EXAMPLE

### Expression of Differentiated Hepatocyte Cell Markers in Cultured Placental Derived Cells

### 9.1. Materials and Methods

### 9.1.1. Cell Isolation and Culture Conditions

Amniotic tissue was obtained from a normal placenta as described in Example 7. The cells were cultured in the Strom and Miki media as presented in Table 3. The cell isolation and culture conditions which differ from those described by Sakuragawa, et al. (Sakuragawa et al., Human Amniotic Epithelial Cells are Promising Transgene Carriers for Allogeneic Cell Transplantation Into Liver. J Hum Genet 45:171-176, 2000.) and also listed in Table 3. The techniques vary in the concentrations of trypsin, digestion times, culture media and media supplements in the basal media (Table 3).

For the isolation of placental-derived cells described in this example, the cells were isolated from the same placenta using the two different techniques. Cells were cultured approximately 10 days in their respective culture media.

TABLE 3
Comparison of Culture medium conditions of Sakuragawa et al and Strom
and Miki.

| | Sakuragawa et al. | Strom and Miki |
|---|---|---|
| Trypsin Conc. | 0.1% | 0.05% |
| Digestion time | 15 min | 30 min × 2 |
| Culture media | RPMI | DMEM (high glucose) |
| Supplement | 10% FBS | 10% FBS |
| | | EGF |
| | | Sodium pyruvate |
| | | 1% non essential amino acid |

The differences in cell isolation and culture may lead to the isolation of cell types different from those isolated and/or propagated using the Sakuragawa technique.

### 9.1.2. RT-PCR

To determine the expression of specific genes in culture real time quantitive real time PCR analysis was performed. RNA was isolated from the cultured cells and examined by quantitive PCR for gene expression. Real time PCR is a process where quantitative analysis of gene expression can be accomplished by doing a normal PCR reaction and measuring the product produced in real time using a fluorescent dye. The dye is in excess in the reaction so that when it interacts with DNA the fluoresces is in proportion to the amount of DNA. It is by this mechanism that one can get a quantitative measurement of the amount of RNA or DNA in the original solution. For RNA quantitation one begins with a reverse transcriptase step to convert RNA into DNA which can then be amplified through regular PCR. These assays are conducted on a real-time PCR machine supplied by Applied Biosystems and a complete protocol for quantitative PCR is supplied as product numbers 4310251 and 4304449. In each case the relative level of expression of the indicated gene is compared to the expression of β-actin, the internal control.

### 9.1.3. Gene Array

To address isolation and culture conditions reported by others, the expression of a large number of genes under the conditions of the present invention and those of Sakuragawa were compared (Table 3). Isolated RNA from the cells were analyzed on a gene array. These arrays contain DNA sequences specific for thousands of genes, such that an analysis of gene expression of several thousand genes can be conducted at one time. Two arrays were run. One with the RNA from the cells isolated and cultures under the methods of Sakuragawa, and another with the cells isolated from the same placenta using the conditions of Strom and Miki (Table 3). Cells were cultured under each condition for two weeks. Cells were scraped and spun down at 1000 rpm for 5 min. The pelleted cells were snap-frozen with liquid nitrogen and stored in −80° C. until analysis. Total RNA was extracted and mRNA was purified to hybridize to DNA microarrays (Affymetrix U133A). Scanned arrays were analyzed with Affymetrix MAS 4.0 software to identify genes which were expressed at different levels between the two conditions.

### 9.2. Results

### 9.2.1. Cell Proliferation

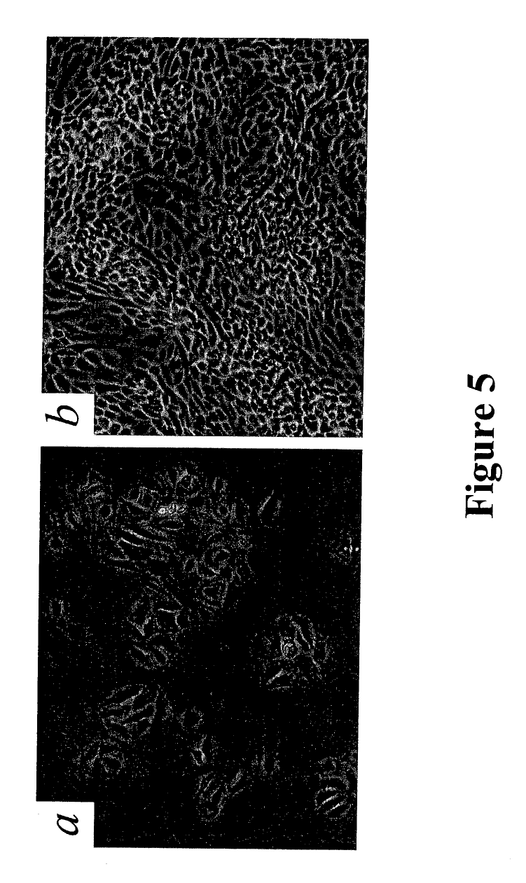

FIG. 5 is a light micrograph showing placental-derived cells isolated from the same placenta using isolation methods and culture medium techniques as described in Sakuragawa et al., (FIG. 5a) or using the isolation method and culture medium techniques of the present invention (FIG. 5b). As indicated, the cells cultured in the media of the present invention proliferate extensively, filling the dish. In contrast, placental-derived cells cultured using the techniques and medium of Sakuragawa et al. support little cell proliferation.

### 9.2.2. Expression of Liver-Specific Markers

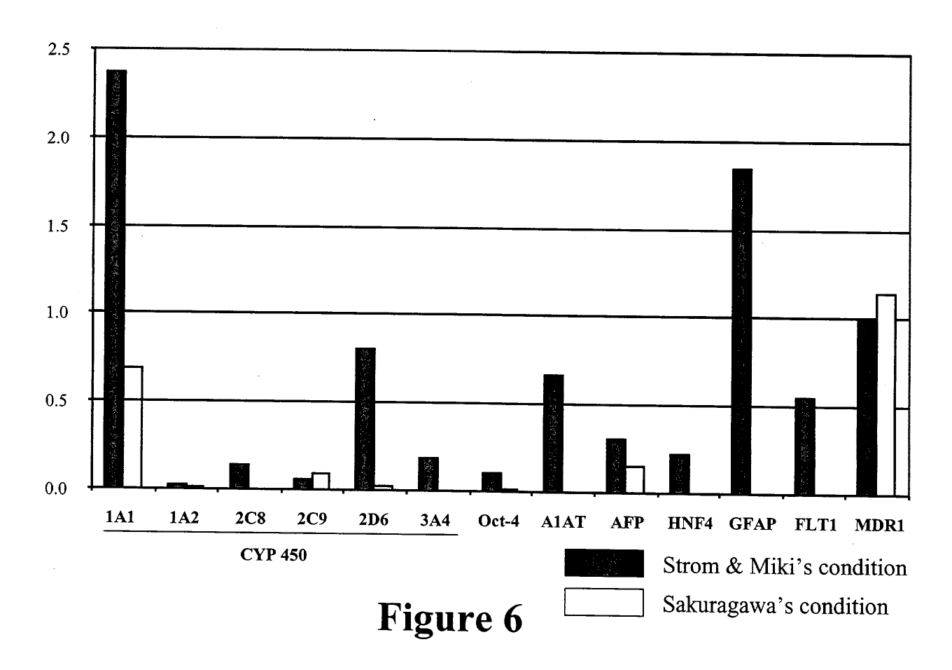

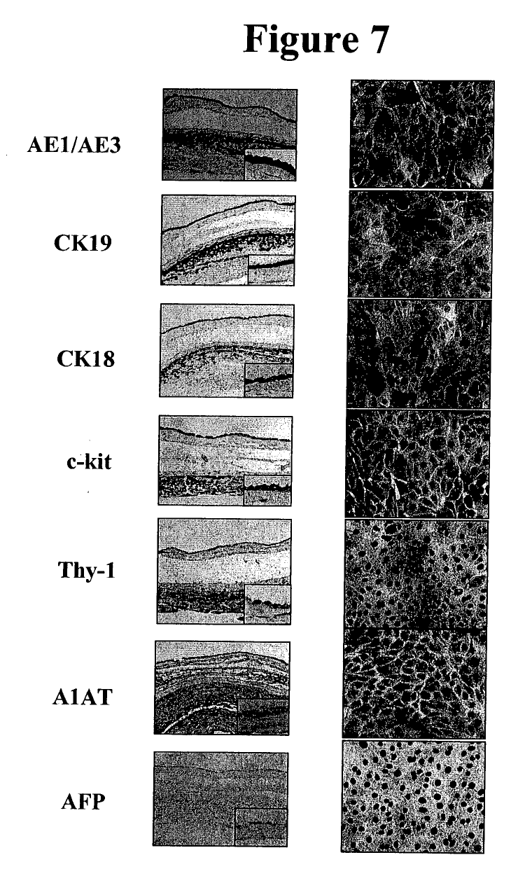

The expression of several liver-specific genes in placental-derived cells cultured using the conditions of Sakuragawa et al. or the methods of the present invention were examined using real time PCR (FIG. 6). The cultured cells were examined for expression of the following liver specific genes, CYP1A1, CYP1A2, CYP2C8, CYP2C9, CYP2D6, CYP3A4, Oct 4, A1AT, AFP, HNF4, GFAP, FLT1, and MDR1. The CYP genes code for drug metabolizing enzymes expressed in the liver. Expression of such liver metabolizing enzymes in cultured placental-derived cells is desirable and will be particularly useful for the generation of hepatocytes for patient transplants, bioartificial liver (BAL) devices or for drug metabolism or toxicology purposes.

Of the 13 different genes examined, only MDR1, or the multidrug resistance gene and CYP2C9 were expressed at similar levels between the culture conditions of Sakuragawa and the conditions of the present invention (FIG. 6). The cultured cells exhibited significant differences in gene expression, in particular, for CYP1A1, CYP 2C8, CYP2D6, and CYP3A4. This disparity suggest that the cells cultured using the method of the present invention demonstrate a far superior ability to differentiate into hepatocytes in comparison to cells isolated using the method of Sakuragawa et al.

Other genes, such as Oct-4, alpha-1 antitrypsin (A1AT), GFAP and FLT-1, are also expressed only under the culture conditions of the present invention (FIG. 6). A1AT and HNF 4 are markers of differentiated hepatocytes. The liver produces and secretes A1AT and HNF4 is a transcription factor required for the maintenance of differentiated liver function.

GFAP is glial fibrillary acid protein, a marker for neuronal glial cells and FLT-1 is a surface receptor expressed on vascular endothelial cells. Both GFAP and FLT-1 are detectable in the placental-derived stem cells isolated and cultured under the conditions of the present invention (FIG. 6). Their expression in placental-derived cells suggest that these cells can differentiate along neuronal and endothelial lineages, as well as towards hepatocyte cell lineages. It is not clear whether these markers, commonly found on different tissue types, are expressed on the same cells or on different cells within our cultures. The presence of markers of differentiated neuroglial cells, differentiated hepatocytes and vascular endothelial cells in the same cultures may indicate that the isolation conditions of the present invention provide a means for the isolation of cells having different differentiation potentials. Alternatively, the media and growth conditions of the present invention may provide a wider range of differentiation potential from the same cell type.

One argument for the isolation of different cell types is the observation of the presence of Oct-4 positive cells only using the isolation and culture conditions of the present invention. Expression of Oct-4 is thought to be restricted to totipotent stem cells such as the ES cells. The presence of Oct-4 in the cell cultures of the present invention, but not that of Sakuragawa et al. indicates the isolation of a different cell type by the isolation conditions of the present invention or the rapid loss of this cell type from cultures obtained using the Sakuragawa technique.

### 9.2.3. Gene Array

A total of 2929 genes were found to be expressed at significantly different levels between the conditions of Strom and Miki and those of Sakuragawa. In this analysis, 885 genes showed an elevated expression under the Strom and Miki protocol while 2044 genes were expressed at lower levels as compared to those under Sakuragawa's conditions. Since the human genome only contains about 30,000 genes and a tissue such as the liver may only express 5,000 total genes, a differential expression of 2923 genes is a large proportion of the total expressed genes. A table of the top fifty genes which were significantly upregulated under the Strom-Miki conditions and the Sakurgawa conditions are summarized in Tables 4 and 5, respectively. Table 6 lists genes which are also significantly upregulated under the Strom and Miki conditions beyond the top fifty listed in Tables 4 and 5. These selected genes contain many important genes for neural, liver, pancreatic and intestinal cells.

The highlighted genes were hepatocyte specific or liver related genes (Table 4 and 5). The genes were ranked by the signal intensity in Log scale, so that a gene shown as 1.0 is expressed at 10 times the level compared to the other condition, and a number of 3 would indicate the gene was expressed at 1000 times (or ten to the third power) different levels between the 2 conditions. It is clear that many of the liver related genes (shaded) are expressed at levels that are 10,000 to 100-million times higher (8.0 in Table 4) under the Strom-Miki conditions as compared to those of Sakuragawa. Seventeen genes (34% of top 50 genes) that were upregulated under the Strom-Miki conditions were liver related, on the other hand only one gene that could be considered as liver related was upregulated in Sakuragawa's conditions.

[image]

TABLE 5
Up-regulated genes in Sakuragawa Medium

| Relate to | Log ratio | Gene description |
|---|---|---|
| A | 10.7 | connective tissue growth factor |
| O | 8.4 | serine proteinase inhibitor, clade E, member 1 |
| | | (SERPINE 1) |
| O | 7.8 | cytolysis inhibitor (CLI) |
| O | 7 | CYR61 |
| O | 6.9 | parathyroid-like protein |
| O | 6.8 | insulin-like growth factor binding protein 7 |
| | | (IGFBP7) |
| O | 6.7 | L-type amino acid transporter 1 |
| L | 6.4 | heptacellular carcinoma novel gene3 protein |
| O | 6.2 | myosin regulatory light chain 2, smooth muscle |
| | | isoform (MYRL2) |
| O | 6.1 | twisted gastrulation (TSG) |
| B | 5.9 | dihydropyrimidinase-like 3 (DPYSL3) |
| B | 5.7 | carboxypeptidase E (CPE) |
| O | 5.6 | hexabrachion (tenascin C, cytotactin) (HXB) |
| O | 5.4 | keratin 17 (KRT17) |
| A | 5.3 | kinesin-like 5 (mitotic kinesin-like protein 1) |
| | | (KNSL5) |
| A | 5.3 | fibroblast growth factor receptor 2(FGFR2) |
| B | 5.2 | GABA-B receptor |
| A | 5.2 | leucine-zipper protein FKSG13 (FKSG13) |
| O | 5.1 | tropomyosin 2 (beta) (TPM2) |
| A | 5 | fibulin 1 (FBLN1), transcript variant C |
| O | 5 | guanylate binding protein 1, interferon-inducible, |
| | | 67kD (GBP1) |
| A | 4.9 | solute carrier family 2, member 3 (SLC2A3) |
| A | 4.9 | G protein-coupled receptor, family C, group 5, |
| | | member B (GPRC5B) |
| O | 4.8 | ovarian beta-A inhibin |
| O | 4.8 | oxytocin receptor (OXTR) |
| O | 4.8 | transgelin (TAGLN) |
| O | 4.8 | transmembrane 4 superfamily member |
| | | (tetraspan NET-2) (NET-2) |
| A | 4.6 | transforming growth factor-beta-2 |
| B | 4.6 | bullous pemphigoid antigen 1 (230240kD) |
| | | (BPAG1) |
| O | 4.6 | CD24 |
| O | 4.5 | ectodermal-neural cortex (with BTB-like domain) |
| | | (ENC1) |
| B | 4.5 | calpain 6 (CAPN6) |
| O | 4.4 | tumor-associated calcium signal transducer 1 |
| | | (TACSTD1) |
| O | 4.4 | lysyl oxidase-like 1 (LOXL1) |
| O | 4.3 | tropomyosin 4 |
| A | 4.3 | eIF4E-transporter (4E-T) |
| A | 4.2 | keratin 14 |
| A | 4.2 | 1,2-cyclic-inositol-phosphate phosphodiesterase |
| | | (ANX3) |
| O | 4.2 | histamine N-methyltransferase (HNMT) |
| O | 4.2 | putative transmembrane protein (NMA) |
| A | 4.2 | latent transforming growth factor beta binding |
| | | protein 3 (LTBP3) |
| B | 4.1 | Kallmann syndrome 1 sequence (KAL1) |
| O | 4.1 | serine proteinase inhibitor, clade H, member 1 |
| | | (SERPINH1) |
| O | 4.1 | wingless-type MMTV integration site family, |
| | | member 11 (WNT11) |
| O | 4.1 | MAD homolog 7 (MADH7) |
| A | 4 | checkpoint suppressor 1 (CHES1) |
| A | 4 | putative endothelin receptor type B-like protein |
| P | 3.9 | cadherin 3, type 1, P-cadherin (placental) (CDH3) |
| O | 3.9 | CD151 antigen (CD151) |
| O | 3.9 | putative integral membrane transporter (LC27) |

TABLE 6
Up-regulated genes in the Present Invention Medium (Selected)

| Log Ratio | Gene description |
|---|---|
| 3 | ATPase, Na + K + transporting, beta 1 polypeptide |
| 2.9 | amylase, alpha 1A; salivary (AMY1A) |
| 2.9 | c-mer proto-oncogene tyrosine kinase (MERTK) |
| 2.8 | activin A receptor, type IIB (ACVR2B) |
| 2.8 | albumin/FL |
| 2.7 | neuritin (LOC51299) |
| 2.7 | UDP glycosyltransferase 1 family, polypeptide A3 (UGT1A3) |
| 2.5 | fibrinogen, gamma polypeptide (FGG), transcript variant |
| | gamma-A |
| 2.4 | colony stimulating factor 1 receptor (CSF1R) |
| 2.4 | cytochrome P450-2A6 (CYP2A6) |
| 2.4 | transforming growth factor, beta receptor III |
| | (betaglycan, 300 kD) (TGFBR3) |
| 2.3 | ATP-binding cassette, sub-family C (CFTRMRP), member |
| | 2 (ABCC2), |
| 2.3 | dipeptidylpeptidase IV (CD26, adenosine deaminase |
| | complexing protein 2) (DPP4) |
| 2.3 | estrogen receptor |
| 2.2 | cytochrome P450IIE1 (ethanol-inducible) |
| 2.1 | cytochrome P450, (CYP2D6) |
| 2.1 | ADP-ribosylation factor-like 4 (ARL4) |
| 2.1 | UDP-N-acetyl-alpha-D-galactosamine:(GalNAc-T7) |
| | (GALNT7) |
| 2 | cytochrome P450IIA3 (CYP2A3) |
| 1.9 | Jak2 kinase (JAK2) |
| 1.9 | acetyl-Coenzyme A acetyltransferase 2 (acetoacetyl |
| | Coenzyme A thiolase) |
| 1.8 | fumarylacetoacetate (FAH) |
| 1.8 | cyclin-E binding protein 1 (LOC51191) |
| 1.7 | insulin-like growth factor 2 (somatomedin A) (IGF2) |
| 1.7 | gamma-aminobutyric acid (GABA) receptor, rho 1 |
| | (GABRR1) |
| 1.6 | FGF receptor 4b |
| 1.5 | cytochrome P450-3A4 (CYP3A4) |
| 1.5 | Similar to solute carrier family 1 (glutamate transporter), |
| | member 7 |
| 1.5 | preproinsulin-like growth factor II (IGF-II) |
| 1.5 | argininosuccinate lyase (ASL) |
| 1.5 | cytochrome P450, (CYP2A7) |
| 1.5 | fucose-1-phosphate guanylyltransferase (FPGT) |
| 1.5 | cytochrome P450, (CYP7B1) |
| 1.5 | inhibin, beta C (INHBC) |
| 1.5 | mitogen-activated protein kinase kinase kinase 12 |
| | (MAP3K12) |
| 1.5 | FLT4 ligand |
| 1.4 | frizzled 1 |
| 1.4 | dopamine receptor D2 (DRD2) |
| 1.4 | ATP-binding cassette, sub-family D (ALD), member 3 |
| | (ABCD3) |
| 1.4 | STAT induced STAT inhibitor-2 (STATI2) |
| 1.4 | mucosal vascular addressin cell adhesion molecule 1 |
| | (MADCAM1) |
| 1.4 | interleukin 1-beta converting enzyme isoform gamma |
| | (IL1BCE) |
| 1.3 | signal transducer and activator of transcription 6, |
| | interleukin-4 induced |
| 1.3 | interleukin-1 beta convertase (IL1BCE) |
| 1.3 | estrogen receptor 1 (ESR1) |
| 1.3 | gastrin (GAS) |
| 1.3 | carboxypeptidase A2 (pancreatic) (CPA2) |
| 1.2 | vascular endothelial growth factor (VEGF) |
| 1.2 | glutathione S-transferase A4 (GSTA4) |
| 1.2 | organic cationic transporter-like 4 (ORCTL4) |
| 1.2 | urokinase-type plasminogen activator receptor |
| 1 | frizzled (Drosophila) homolog 1 (FZD1) |
| 0.9 | adipose differentiation-related protein |
| 0.9 | keratin 19 (KRT19) |
| 0.9 | neuroendocrine secretory protein 55 (NESP55) |
| 0.8 | thiopurine methyltransferase (TPMT) |

### 10. EXAMPLE

### Differentiation Media Promotes Hepatocyte Differentiation in Cultured Placental-Derived Cells

### 10.1. Materials and Methods

Placental-derived cells were isolated as described in Example 7 and cultured using basal culture conditions found in Table 3 for cell plating and expansion of the cells for 10-14 days. The cells were cultured for either 7-10 days or until the cultures grew to confluence. The cells were trypsinized and reseeded in 6 well plates. The cells were subjected to different culture conditions, as indicated in each Figure, having varying growth factors supplementing the DMEM or MEM based medium. Placental-derived cells were cultured for an additional 14 days. At the end of 14 days, the cells were evaluated for the expression of human albumin, CYP3A4, A1AT, or C/EBP-alpha.

RT-PCR was also run on RNA isolated from the cells and as described in Example 6.

### 10.2. Results

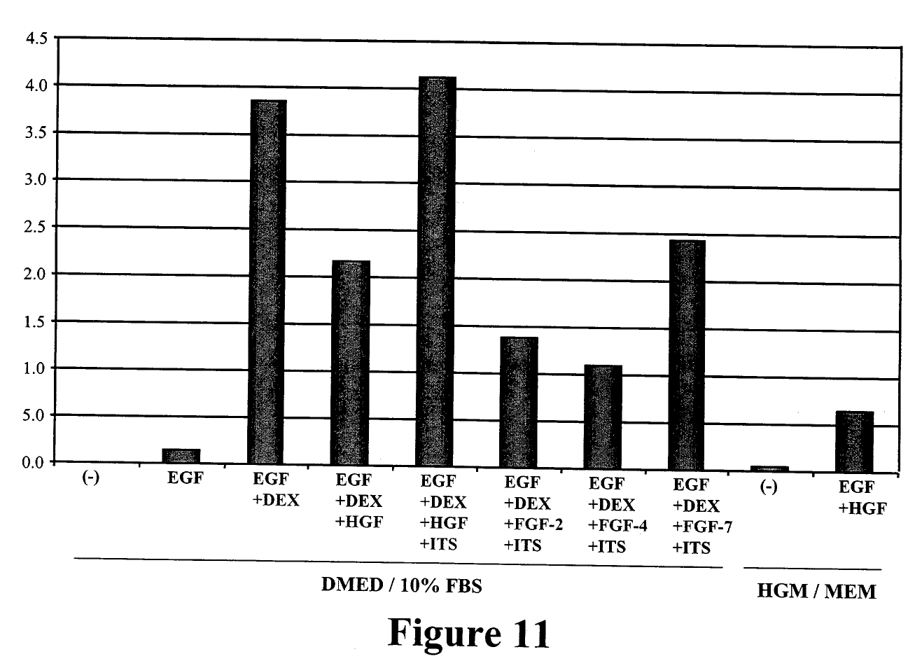

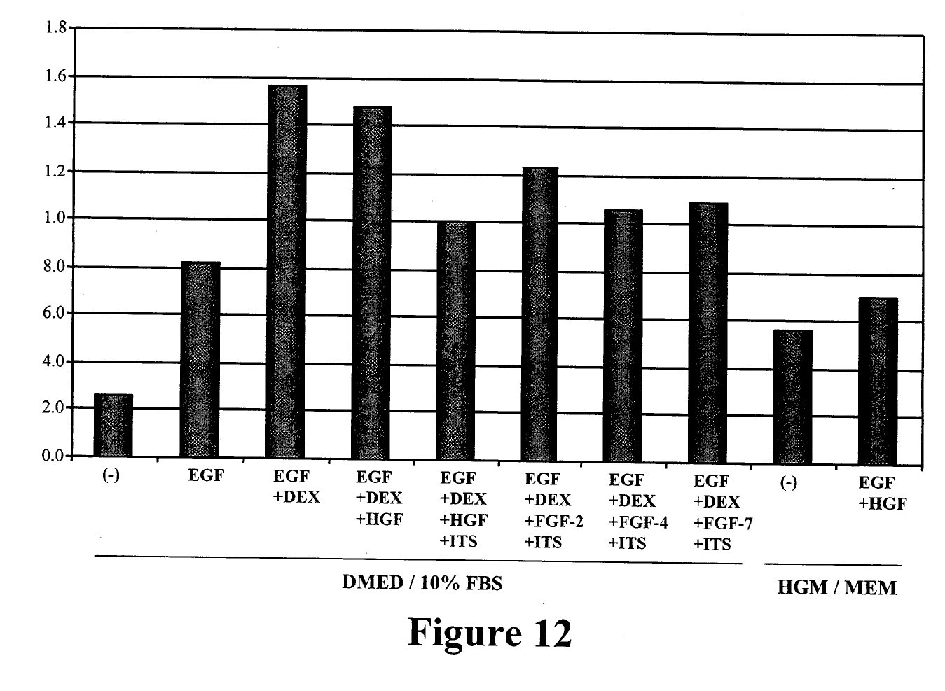

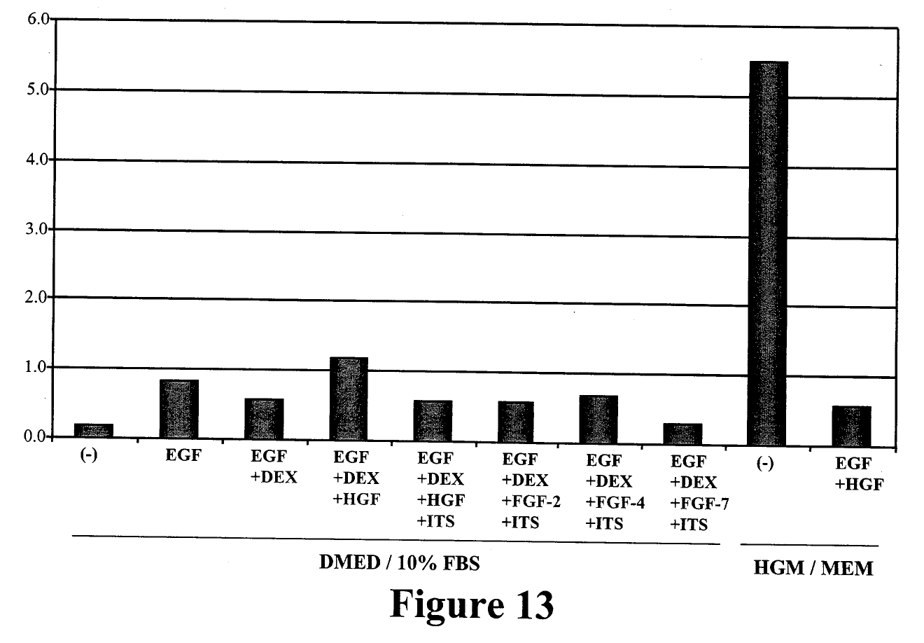

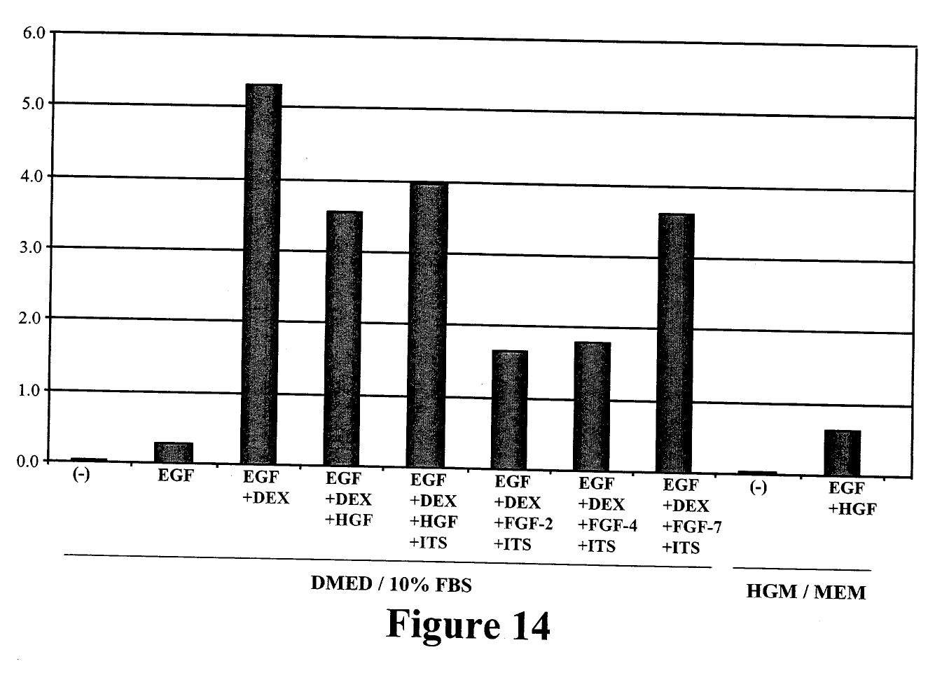

A panel of media supplemented with various growth factors and/or combinations of growth factors were used to culture the placental-derived cells to identify optimal culture media for enhanced expression of liver specific genes. The cultured cells were analyzed for expression of human albumin (FIG. 11), CYP3A4 (FIG. 12), A1AT (FIG. 13) and C/EBP-alpha (FIG. 14). The results indicate that under certain culture conditions the expression of albumin, CYP3A4, A1AT and C/EBP alpha increase considerably over the initial values reported in FIG. 6. In particular, the inclusion of EGF and dexamethasone (Dex) was shown to enhance liver specific gene expression. At least at these time points in culture, the additional supplementation of the media with HGF, hepatocyte growth factor, or the fibroblast growth factors (FGF) 2,4, or 7 did not enhance liver specific gene expression. The data presented in FIGS. 11-14 indicate that modification of the culture conditions from the basal growth media listed in Table 3 to the differentiation media conditions indicated in each of the Figures can enhance the expression of liver specific genes. These results suggest that liver specific gene expression is enhanced by the use of the differentiation media described herein. While HGF and the fibroblast growth factors did not appear to enhance differentiation along the hepatocyte pathway, these growth factors may promote differentiation of cultured cells to other cell types such as neuronal, pancreatic or muscle cell differentiation.

### 11. EXAMPLE

### Cultured Placental-Derived Hepatocytes Demonstrate Metabolic Function

### 11.1. Materials and Methods

Human hepatocytes were cultured using the conditions of Strom et al., 1996 methods in Enzymology 272:388-401.

An EROD assay which measures the conversion of ethoxy-resorufin to hydroxyresorufin was used to detect expressin of CYP1A1 or CYP1A2 in liver by the enzyme CYP1A1 or 1A2. Kelley et al, 2000 J. Biomolecular Screening 5:249-253.

The expression of CYP3A4 in the liver was measured as the specific conversion of testosterone to the 6-beta-hydroxy metabolite. Kostrubsky et al., 199, Drug Metab. dispos 27:887-894.

Uptake of Indocyanine Green (ICG) is another clinical test that may be utilized to assay for liver function. In patients, ICG is injected into the blood stream and as it passes through the liver the dye is taken up by transport proteins specific to the liver. The transporter proteins involved in the uptake of ICG are called OATP (organic anion transporter protein) and a liver specific organic anion transporter (LST).

### 11.2. Results

Normally analysis of CYP1A1 and CYP3A4 activity in human hepatocytes is accomplished by measuring the ability of the cells to metabolize drugs or specific compounds which are substrates for different CYP450 genes. The levels of these enzymatic processes in AE-derived hepatocytes were evaluated using the EROD assay, presence of 6-beta-hydroxy metabolite, and uptake of indocyanine green.

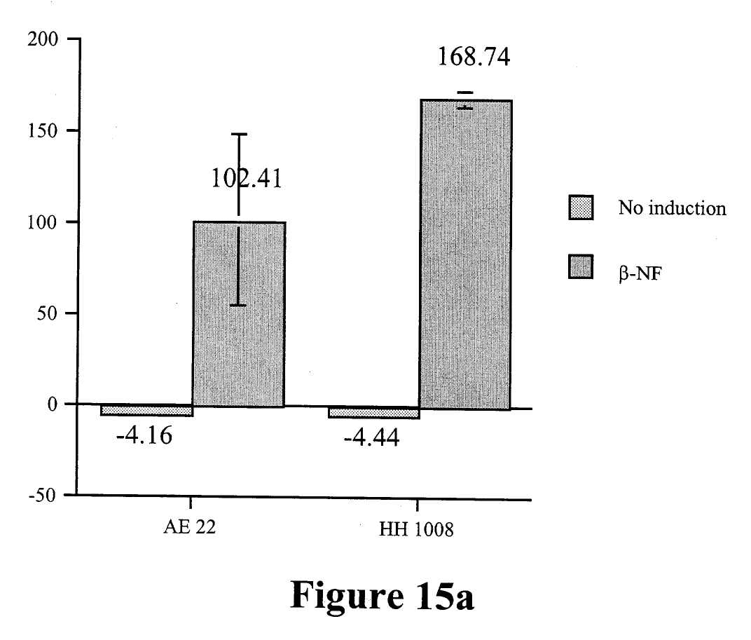

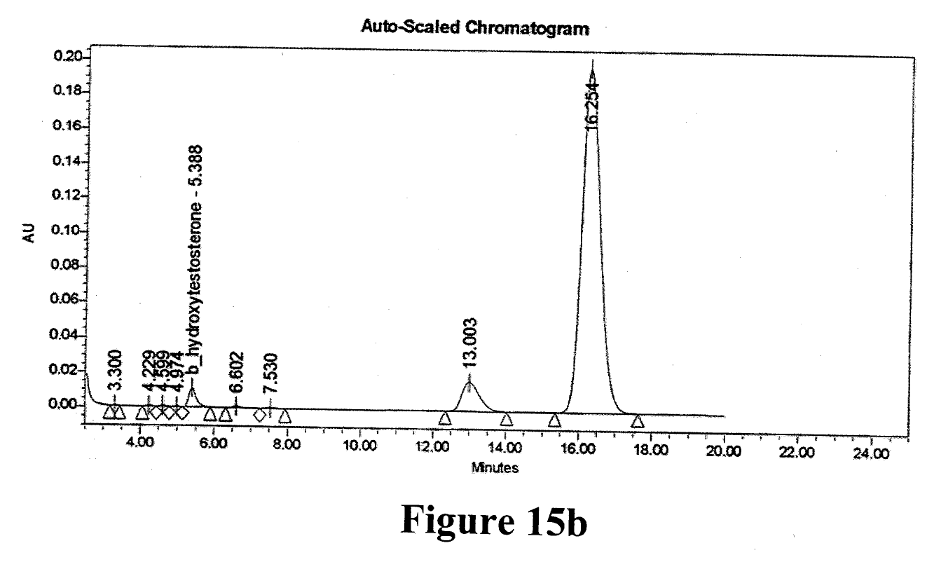

Data presented in FIG. 15 shows the results of the ethoxyresorufin assay (FIG. 15A) and the metabolism of testosterone (FIG. 15B). As shown in FIG. 15A, AE-derived hepatocytes metabolize ethoxyresorufin. For comparison, the EROD assay was also performed on authentic human hepatocytes isolated from a donor liver not used for whole organ transplantation. As with human liver (HH 1008), the placental-derived hepatocytes do not express much enzymatic activity under basal conditions. With both the hepatocytes and the placental-derived hepatocytes, EROD activity is induced by prior exposure of the cells to beta-naphthoflavone (BNF). Beta-Naphthoflavone was chosen for this study based on its ability to stimulate CYP1A1/2 expression in the liver and in cultured hepatocytes. The data indicate that the expression of CYP1A1/2 in the placental-derived hepatocytes is equal to approximately 60% of the activity seen in authentic human hepatocytes.

The data presented in FIG. 15B shows high pressure liquid chromatographic (HPLC) separation of testosterone metabolites generated in placental-derived hepatocytes. There is a clearly production of 6-beta-hydroxytestosterone by placental-derived hepatocytes. The data presented in FIG. 15 demonstrate unequivocally that the placental-derived hepatocytes not only express RNA for the specific P450 genes, but that the cells actually translate the protein and make active drug metabolizing enzymes. The presence of such metabolic functions confirm the usefullness of such cells for drug metabolism or toxicology studies, artificial liver devices or for clinical hepatocyte transplants.

The uptake of ICG by placental-derived hepatocytes was also examined to determine the placental-derived cells exhibited hepatocyte function. 13.9% of the placental-derived hepatocytes show uptake of ICG in comparison to 46.4% in human hepatocytes. These data indicate the presence of liver specific drug and chemical transporters on the placental-derived hepatocytes and further establish the utility of the placental-derived hepatocytes for drug metabolism and toxicology studies as well as artificial liver devices and hepatocyte transplants.

### 12. EXAMPLE

### Transplantation of Cultured Placental-Derived Cells into Mouse Liver and Differentiation to Human Hepatocytes

### 12.1. Materials and Methods

Placental-derived cells were transplanted into the liver via the spleen. Because of bleeding difficulties following direct transplantation of cells into liver or portal vein, it has been established that approximately 50% of the cells transplanted into the spleen will translocate to the liver within 5 minutes (Ponder et al., “Mouse hepatocytes migrate to liver parachrome function indefinitely after intrasplenic transplantation.” Genetics 88:1217 (1991). Once in the liver, transplanted hepatocytes incorporate into hepatic plates and survive long-term. Placental-derived cells were used as hepatocytes and the fate of the transplanted cells were followed. Pictures were taken one month following transplantation.

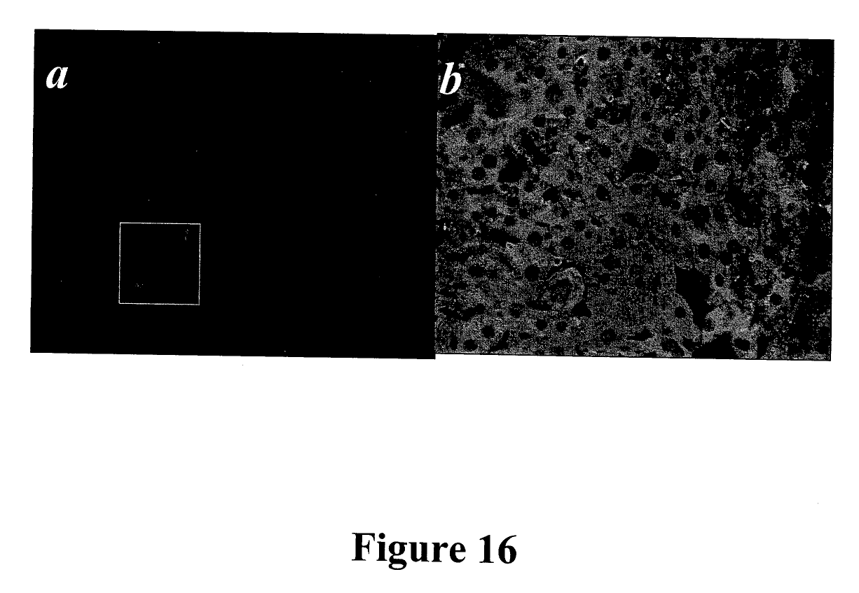

Placental-derived cells were transplanted in place of hepatocytes and the fate of the transplanted cells were followed in the liver. Pictures in FIGS. 16-18 were taken 1 month following the transplantation of two million cells into the spleen. In FIG. 16 only, placental-derived cells were first infected with an adenovirus vector containing GFP to label the cells prior to transplantation. At the time of transplantation >85% of the placental-derived cells were labeled with the green fluorescent protein. Recipient animals were SCID or Rag-2 knock out animals. These mouse strains are immunocompromised and are regularly used for investigations of the transplantation of human tissues or cells because the animals do not readily reject the foreign tissue/cells.

### 12.2. Results

As shown in FIGS. 16-18, placental-derived cells translocate to the liver from the spleen, integrate into hepatic plates and express the morphology of hepatocytes and genes associated with normal liver. In the experiments summarized in FIG. 16, placental-derived cells were labeled with a viral vector expressing Green Fluorescent Protein (GFP) and transplanted the cells into the liver via the spleen. As shown in FIG. 16, labeled cells can be observed in sections of the liver of these animals. There are five labeled cells in the liver section (FIG. 16a). FIG. 16b shows a 400×micrograph of the two labeled cells shown in FIG. 16a. The fluorescently labeled cells exhibit the morphology of normal hepatocytes which have been incorporated into hepatic plates. Cells which do not incorporate into hepatic plates die and are rapidly removed from the liver my macrophages within 3-7 days, so the results presented in FIGS. 16-18 represent only those cells which have become stably incorporated into the mouse liver. The frequency of integration of the placental-derived cells into the liver can be calculated from the number of labeled cells recovered from the liver. The frequency of integration is very high for placental-derived hepatocytes as compared to normal hepatocytes. In published reports, following the transplantation of hepatocytes integration frequencies range from 0.1% to 10% of the transplanted cells. The integration of placental-derived hepatocytes is approximately 51% of the transplanted cells. These data indicate that the placental-derived cells will be useful for hepatocyte transplantation studies.

Transplants of actual human hepatocytes into immunocompromised mice provide virtually identical results to that shown here following the transplantation of the placental stem cells (Dandri, M, Burda, M, Torok, E, Pollok, J M, Iwanska, A, Sommer, G, Rogiers, X, Rogler, C E, Gupta, S, Will, H, Greten, H., Petersen, J. Repopulation of mouse liver with human hepatocytes and In Vivo infection with hepatitis B virus. Hepatol. 33:981-988, 2001, and Mercer, D F, Schiller, D E, Elliott, J F, Douglas, D N, Hao, C., Rinfret, A, Addison, W R, Fischer, K P, Churchill, T A, Lakey, J R T, Tyrell D L J, Kneteman, N M. Hepatitis C virus replication in mice with chimeric human livers. Nature Med. 7:927-933, 2001). These data indicate that the transplanted stem cells mature to human hepatocytes in the liver of the recipient.

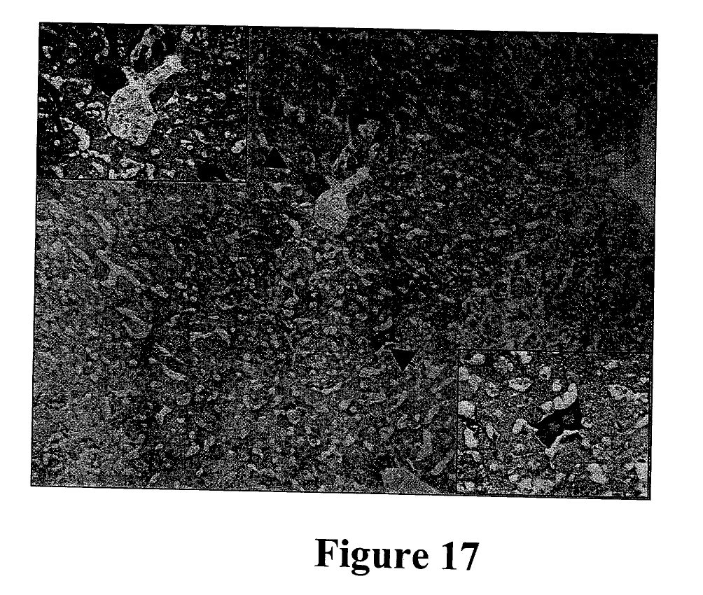

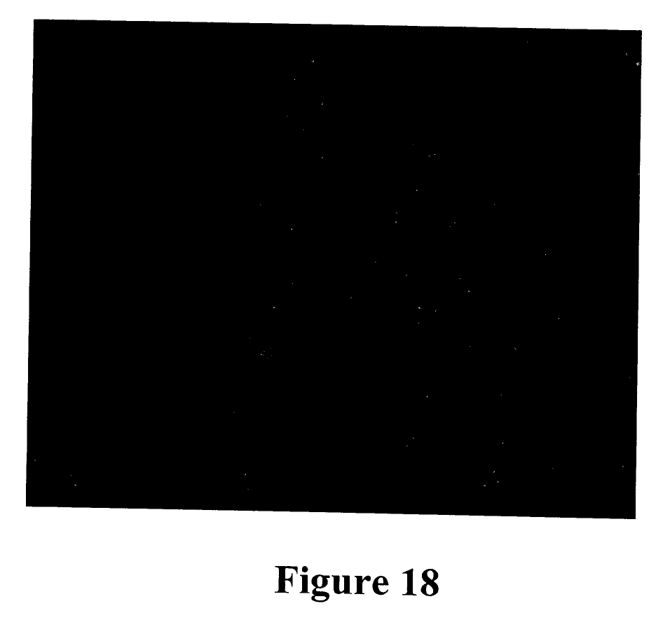

Isolated placental-derived cells were also transplanted into the liver of immunodeficient mice. At I month following transplantation, animals were sacrificed and sections were made of the liver of transplanted animals. Liver sections examined for transplanted cells with antibodies to human alpha-1 antitrypsin (FIG. 17) or human albumin (FIG. 18). Data presented in FIGS. 17 and 18 clearly show cells with the morphology of hepatocytes in the liver sections which react with antibodies to human A1AT (FIG. 17) or human albumin (FIG. 18). These data confirm that human placental-derived cells transplanted into the liver of immunodeficient mice integrate into the hepatic plates, have the morphology of normal human hepatocytes and express genes usually expressed in normal liver.

### 13. EXAMPLE

### Differentiation of Placental-Derived Stem Cells into Neuronal and Vascular Endothelial Cells

### 13.1. Materials and Methods

Placental-derived cells were cultured in the presence of FGF-4 (10 ng/ml) for approximately 14 days. Immunohistochemical analysis of the expression of the different genes was conducted with antibodies specific to the human proteins.

Cells were cultured for approximately 10 days in both growth mode and plated on dishes coated with Matrigel™ (20T on 100% u/u). Placental-derived cells were cultured on Matrigel as also disclosed in Grant et al and Kazuya et al. (Grant, D S, Lelkes, P I, Fukuda, K, Kleinman H K. Intracellular mechanisms involved in basement membrane induced blood vessel differentiation in vitro. In Vitro Cell Dev. Biol 27A: 327-336, 1991; and Kazuya, M and Kinsella, J L, Reorganization of endothelial cord-like structures on basement membrane complex (Matrigel): involvement of transforming growth factor beta 1. J. Cell Physiol 161: 267-276, 1994.)

### 13.2. Results

FIG. 19 shows that cultured placental-derived cells express GFAP, glial fibrillary acidic protein, a marker of oligodendrocytes, beta-tubulin III, a marker for astrocytes, and CNP, a marker for neurons. In the presence of FGF-4, placental-derived cells take on a neuronal shape and begin to express various neuronal markers. These results indicate that the cultured placental-derived cells are multipotent, that is, they can differentiate along the neuronal, oligodendrocyte and astrocytic lineages.

On a culture substrate called matrigel, vascular endothelial cells aggregate into web-like formations and form tubules with open lumens (FIG. 20). These morphologic changes indicate the first steps in the development of vascular channels. Cells on matrigel and observed web-like formation reminiscent of authentic vascular endothelial cells are shown in FIG. 20. At higher power (400×) the elongated capillary-like structure is clearly observed (FIG. 20). A transmission electron micrograph (4,000×) shows the rudimentary formation of a vascular channel (FIG. 20). These data indicate that cultured AE-derived cells differentiate along an endothelial cell pathway and can be used as stem cells for the formation, reconstruction or repair of the human vascular system.

### 14. EXAMPLE

### Differentiation of Placental-Derived Stem Cells into Pancreatic Cells

### 14.1. Materials and Methods

Placental-derived cells were maintained in standard growth media for 7 days and then trypsinized and seeded on cultures previously coated with matrigel (MG), a commercially available form of basement membrane proteins. Cultures were coated with 20% (v/v; matrigel to media) or 100% matrigel with essentially identical results. Data from the 20% matrigel experiments are shown here. Cells were seeded on plates previously coated with MG and were cultured an additional 14 days in standard media supplemented with Dexamethasone (0.1 micromolar) and the standard concentrations of ITS. After 10 days the cells were lysed and RNA was isolated, and a one-stem reverse transcriptase-PCR analysis was conducted with PCR primers specific for Pax 6, PDX-1 Nkx2.2, insulin, glucagons and the internal control beta-actin.

### 14.2. Results

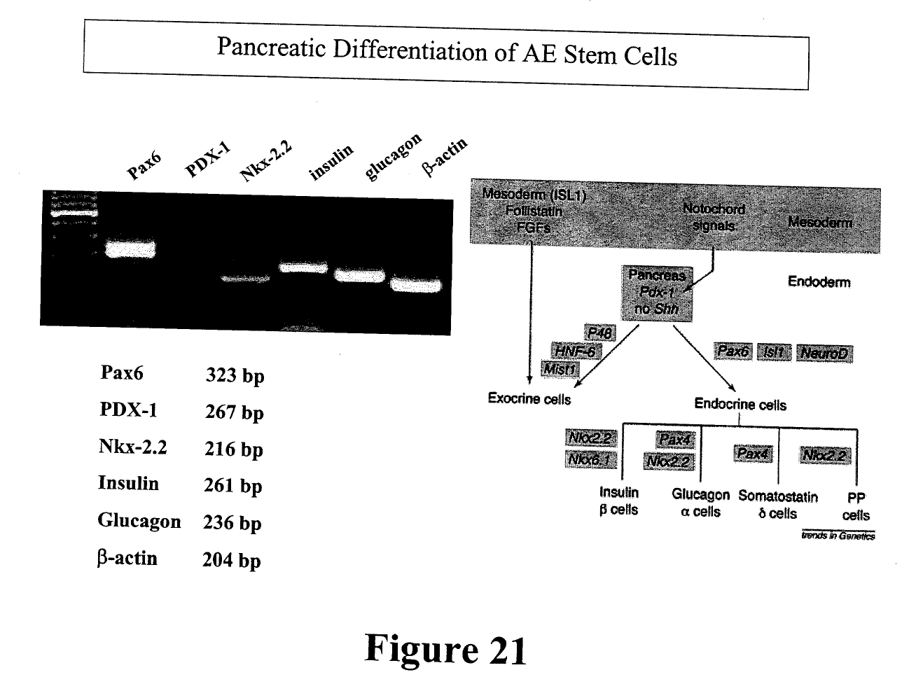

To investigate the potential for differentiation into pancreatic cells, placental-derived cells were cultured in basal medium for 7 days and examined for the expression of insulin and the transcription factors necessary for pancreatic beta cell differentiation, e.g. Pax6 and Pdx1. FIG. 21 shows that the culture cells express Pax6, Pdx1 and insulin. Although the expression of Pdx1 is weak, the expression of insulin is quite strong. Placental stem cells treated in this manner express markers of pancreatic differentiation including PDX-1, Pax 6 and Nkx2.2 that promote endocrine cell differentiation as well as markers specific to beta cells (insulin) or alpha cells (glucagon). These results indicate that the placental stem cells have the capacity to differentiate to pancreatic cells. Expression of insulin in these cultured cells indicates that they have the potential to be used as pancreatic islet cells. These cells may secrete insulin for use towards the treatment of diabetes. The expression of pancreatic markers for alpha, as well as beta cells suggest that the regeneration of most or all cell types of the pancreas may be possible with the placental stem cells of the present invention.

The present invention is not to be limited in scope by the specific embodiments described herein. Indeed, various modifications of the invention in addition to those described herein will become apparent to those skilled in the art from the foregoing description and accompanying Figures. Such modifications are intended to fall within the scope of the appended claims. Various references are cited herein, the disclosure of which are incorporated by reference in their entireties.

We claim:

1. A composition comprising a placental derived stem cell, wherein the cell expresses at least one marker selected from the group: c-kit, Thy-1, OCT-4, SOX2, hTERT, SSEA1, SSEA3, SSEA4, TRA1-60 and TRA1-81.

2. The composition of claim 1, which is deposited under ATCC Accession No. XXXX

3. A pharmaceutical composition of claim 1, wherein said stem cell is genetically engineered to produce a therapeutic protein in an amount effective to treat a subject.

4. A pharmaceutical composition comprising the composition of claim 1 in an amount effective to treat a subject with a liver disease, a vascular disease, a disease of nervous tissue, or a pancreatic disease.

5. A method wherein the stem cell of claim 1 is cultured in a dexamethasone, EGF, or TGF-alpha containing media under appropriate conditions and for a sufficient period of time to induce hepatocyte differentiation.

6. A hepatocyte obtained from the process of claim 5 wherein the cell expresses at least one marker selected from the group: albumin, CYP3A4, A1AT, HNF1, HNF4 and C/EBP-alpha.

7. A pharmaceutical composition comprising the hepatocytes obtained from the process of claim 5 in an amount effective to treat a subject with a liver disease.

8. The hepatocytes obtained from the process of claim 5 wherein said hepatocytes are used in determining whether a test agent is toxic to a hepatic cell, comprising contacting said hepatocytes with the test agent for a time sufficient for a toxic effect on the cell to be detected, and determining the toxic effect on the hepatocytes.

9. The hepatocytes obtained from the process of claim 5 wherein said hepatocytes are used in determining a metabolic product of a test agent, comprising contacting said hepatocytes with the test agent for a time sufficient for the test agent to be metabolized, and detecting the presence of a metabolized product.

10. A method wherein the stem cells of claim 1 are cultured on matrigel under appropriate conditions and for a sufficient period of time to induce vascular endothelial cell differentiation.

11. A pharmaceutical composition comprising the vascular endothelial cells obtained from the process of claim 10 in an amount effective to treat a subject with a vascular disease.

12. The vascular endothelial cells obtained from the process of claim 10 wherein said vascular endothelial cells are used in determining whether a test agent is toxic to a vascular endothelial cell, comprising contacting said vascular endothelial cells with the test agent for a time sufficient for a toxic effect on the cells to be detected, and determining the toxic effect on the vascular endothelial cells.

13. The vascular endothelial cells obtained from the process of claim 10 wherein said vascular endothelial cells are used in determining a metabolic product of a test agent, comprising contacting said vascular endothelial cells with the test agent for a time sufficient for the test agent to be metabolized, and detecting the presence of a metabolized product.

14. A method wherein the stem cells of claim 1 are cultured in a dexamethasone, ITS and matrigel containing media under appropriate conditions and for a sufficient period of time to induce pancreatic cell differentiation.

15. A pancreatic cell obtained from the process of claim 14 wherein the cell expresses at least one marker selected from the group, Pax6, Pdx1 and insulin.

16. A pharmaceutical composition comprising the pancreatic cells obtained from the process of claim 14 in an amount effective to treat a subject with a pancreatic disease.

17. The pancreatic cells obtained from the process of claim 14 wherein said pancreatic cells are used in determining whether a test agent is toxic to a pancreatic cell, comprising contacting said pancreatic cells with the test agent for a time sufficient for a toxic effect on the cells to be detected, and determining the toxic effect on the pancreatic cells.

18. The pancreatic cells obtained from the process of claim 14 wherein said pancreatic cells are used in determining a metabolic product of a test agent, comprising contacting said pancreatic cells with the test agent for a time sufficient for the test agent to be metabolized, and detecting the presence of a metabolized product.

19. A method wherein the stem cells of claim 1 are cultured in a FGF-4 containing media under appropriate conditions and for a sufficient period of time to induce differentiation into cells of the nervous tissue.

20. A nervous tissue cell obtained from the process of claim 19, wherein the cell expresses at least one marker from the group: GFAP,FLT1, CNP, and beta-tubulin III.

21. A pharmaceutical composition comprising the nervous tissue cells obtained from the process of claim 19 in an amount effective to treat a subject with a disease of the nervous tissue.

22. The nervous tissue cells obtained from the process of claim 19 wherein said nervous tissue cells are used in determining whether a test agent is toxic to a nervous tissue cell, comprising contacting said nervous tissue cells with the test agent for a time sufficient for a toxic effect on the cells to be detected, and determining the toxic effect on the nervous tissue cells.

23. The nervous tissue cells obtained from the process of claim 19 wherein said nervous tissue cells are used in determining a metabolic product of a test agent, comprising contacting said nervous tissue cells with the test agent for a time sufficient for the test agent to be metabolized, and detecting the presence of a metabolized product.

24. A process deriving a humanized organ comprising transplantation of placental-derived stem cells into an animal organ, wherein said stem cells regenerate and repopulate the animal organ to produce a humanized organ.

25. A humanized organ obtained from the process of claim 24.
